(19) United States
(12) Patent Application Publication
(10) Pub. No.: US 2020/0123115 A1
(43) Pub. Date: Apr. 23, 2020

(54) COMPOUNDS HAVING MULTIMODAL ACTIVITY AGAINST PAIN

(71) Applicant: ESTEVE PHARMACEUTICALS, S.A., Barcelona (ES)

(72) Inventors: Susana YENES-MINGUEZ, Molins de Rei (ES); Carmen ALMANSA-ROSALES, Barcelona (ES)

(21) Appl. No.: 16/619,243

(22) PCT Filed: Jun. 25, 2018
(86) PCT No.: PCT/EP2018/066885

§ 371 Date: Dec. 4, 2019

(30) Foreign Application Priority Data
Jun. 26, 2017 (EP) 17382395.6

(51) Int. Cl. C07D 239/34; C07D 239/26; C07D 237/08; C07D 239/38; C07D 239/42; C07D 213/30; C07D 213/64; C07D 277/24; C07D 333/16; C07D 409/12; C07D 403/12; C07D 409/14

(52) U.S. Cl. CPC C07D 239/34; C07D 239/26; C07D 237/08; C07D 239/38; C07D 239/42; C07D 409/14; C07D 213/64; C07D 277/24; C07D 333/16; C07D 409/12; C07D 403/12; C07D 213/30

(57) ABSTRACT

The present invention relates to a compound of formula (I):

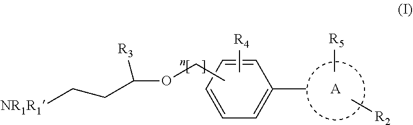

wherein the meanings for the various substituents are as disclosed in the description, having dual pharmacological activity towards both the α2δ subunit, in particular the α2δ-1 subunit, of the voltage-gated calcium channel and the NET receptor, to processes of preparation of such compounds, to pharmaceutical compositions comprising them, and to their use in therapy, in particular for the treatment of pain.

### FIELD OF THE INVENTION

The present invention relates to compounds having dual pharmacological activity towards both the α₂δ subunit of the voltage-gated calcium channel, and the noradrenaline transporter (NET), to processes of preparation of such compounds, to pharmaceutical compositions comprising them, and to their use in therapy, in particular for the treatment of pain.

### BACKGROUND OF THE INVENTION

The adequate management of pain constitutes an important challenge, since currently available treatments provide in many cases only modest improvements, leaving many patients unrelieved (Turk, D. C., Wilson, H. D., Cahana, A.; 2011; Lancet; 377; 2226-2235). Pain affects a big portion of the population with an estimated prevalence of 20% and its incidence, particularly in the case of chronic pain, is increasing due to the population ageing. Additionally, pain is clearly related to comorbidities, such as depression, anxiety and insomnia, which leads to important productivity losses and socio-economical burden (Goldberg, D. S., McGee, S. J.; 2011; BMC Public Health; 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi et al., Pharmacol. Rev. 2015 67:821-70). The VGCC are assembled through interactions of different subunits, namely α₁ (Caᵥα₁), β (Caᵥβ) α₂δ (Caᵥα₂δ) and γ (Caᵥγ). The α₁ subunits are the key porous forming units of the channel complex, being responsible for the Ca²⁺ conduction and generation of Ca²⁺ influx. The α₂δ, β, and γ subunits are auxiliary, although very important for the regulation of the channel, since they increase the expression of the α₁ subunits in the plasma membrane as well as modulate their function, resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N-(Caᵥ2.2), P/Q-(Caᵥ2.1), and R-(Caᵥ2.3) types, depending on the channel forming Cava subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo, Neurotherapeutics. 2009 6:679-92; Zamponi et al., 2015 supra; Neumaier et al., Prog. Neurobiol. 2015 129:1-36.). A large body of data has clearly indicated that VGCC are implicated in mediating various disease states including pain processing. Drugs interacting with the different calcium channel subtypes and subunits have been developed. Current therapeutic agents include drugs targeting L-type Caᵥ1.2 calcium channels, particularly 1,4-dihydropyridines, which are widely used in the treatment of hypertension. T-type (Caᵥ3) channels are the target of ethosuximide, widely used in absence epilepsy. Ziconotide, a peptide blocker of N-type (Caᵥ2.2) calcium channels, has been approved as a treatment of intractable pain. (Perret and Luo, 2009, supra; Vink and Alewood, Br J Pharmacol. 2012 167:970-89.).

The Caᵥ1 and Caᵥ2 subfamilies contain an auxiliary α₂δ subunit, which is the therapeutic target of the gabapentinoid drugs of value in certain epilepsies and chronic neuropathic pain. To date, there are four known α₂δ subunits, each encoded by a unique gene and all possessing splice variants. Each α₂δ protein is encoded by a single messenger RNA and is posttranslationally cleaved and then linked by disulfide bonds. Four genes encoding α₂δ subunits have now been cloned. α₂δ-1 was initially cloned from skeletal muscle and shows a fairly ubiquitous distribution. The α₂δ-2 and α₂δ-3 subunits were subsequently cloned from brain. The most recently identified subunit, α₂δ-4, is largely nonneuronal. The human α₂δ-4 protein sequence shares 30, 32 and 61% identity with the human α₂δ-1, α₂δ-2 and α₂δ-3 subunits, respectively. The gene structure of all α₂δ subunits is similar. All α₂δ subunits show several splice variants (Davies et al., Trends Pharmacol Sci. 2007 28:220-8; Dolphin A C, Nat Rev Neurosci. 2012 13:542-55, Biochim Biophys Acta. 2013 1828:1541-9.).

The Caᵥα₂δ-1 subunit may play an important role in neuropathic pain development (Perret and Luo, 2009, supra; Vink and Alewood, 2012, supra). Biochemical data have indicated a significant Caᵥα₂δ-1, but not Caᵥα₂δ-2, subunit upregulation in the spinal dorsal horn, and DRG (dorsal root ganglia) after nerve injury that correlates with neuropathic pain development. In addition, blocking axonal transport of injury-induced DRG Caᵥ26-1 subunit to the central presynaptic terminals diminishes tactile allodynia in nerve injured animals, suggesting that elevated DRG Caᵥ26-1 subunit contributes to neuropathic allodynia.

The Caᵥα₂δ-1 subunit (and the Caᵥα₂δ-2, but not Caᵥα₂δ-3 and Caᵥα₂δ-4, subunits) is the binding site for gabapentin which has anti-allodynic/hyperalgesic properties in patients and animal models. Because injury-induced Caᵥα₂δ-1 expression correlates with neuropathic pain development and maintenance, and various calcium channels are known to contribute to spinal synaptic neurotransmission and DRG neuron excitability, injury-induced Caᵥα₂δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα₂δ-1 subunit can block nerve injury-induced Caᵥα₂δ-1 upregulation and prevent the onset of allodynia and reserve established allodynia.

As mentioned above, the α₂δ subunits of VGCC form the binding site for gabapentin and pregabalin, which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα₂δ subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009, supra; Vink and Alewood, 2012, supra, Zamponi et al., 2015, supra).

It is also known that Noradrenaline (NA), also called norepinephrine, functions in the human brain and body as a hormone and neurotransmitter. Noradrenaline exerts many effects and mediates a number of functions in living organisms. The effects of noradrenaline are mediated by two distinct super-families of receptors, named alpha- and beta-adrenoceptors. They are further divided into subgroups exhibiting specific roles in modulating behavior and cognition of animals. The release of the neurotransmitter noradrenaline throughout the mammalian brain is important for modulating attention, arousal, and cognition during many behaviors (Mason, S. T.; Prog. Neurobiol.; 1981; 16; 263-303).

The noradrenaline transporter (NET, SLC6A2) is a monoamine transporter mostly expressed in the peripheral and central nervous systems. NET recycles primarily NA, but also serotonin and dopamine, from synaptic spaces into presynaptic neurons. NET is a target of drugs treating a variety of mood and behavioral disorders, such as depression, anxiety, and attention-deficit/hyperactivity disorder (ADHD). Many of these drugs inhibit the uptake of NA into the presynaptic cells through NET. These drugs therefore increase the availability of NA for binding to postsynaptic receptors that regulate adrenergic neurotransmission. NET inhibitors can be specific. For example, the ADHD drug atomoxetine is a NA reuptake inhibitor (NRI) that is highly selective for NET. Reboxetine was the first NRI of a new antidepressant class (Kasper et al.; Expert Opin. Pharmacother.; 2000; 1; 771-782). Some NET inhibitors also bind multiple targets, increasing their efficacy as well as their potential patient population.

For instance, the antidepressants venlafaxine and duloxetine are dual reuptake inhibitor of serotonin and NA that targets both NET and the serotonin transporter (SERT, SLC6A4). Duloxetine has been licensed for major depressive disorder, generalised anxiety disorder, diabetic peripheral neuropathic pain, fibromyalgia and chronic musculoskeletal pain.

Endogenous, descending noradrenergic fibers impose analgesic control over spinal afferent circuitry mediating the transmission of pain signals (Ossipov et al.; J. Clin. Invest.; 2010; 120; 3779-3787). Alterations in multiple aspects of noradrenergic pain processing have been reported, especially in neuropathic pain states (Ossipov et al., 2010; Wang et al.; J. Pain; 2013; 14; 845-853). Numerous studies have demonstrated that activation of spinal α₂-adrenergic receptors exerts a strong antinociceptive effect. Spinal clonidine blocked thermal and capsaicin-induced pain in healthy human volunteers (Ossipov et a., 2010). Noradrenergic reuptake inhibitors have been used for the treatment of chronic pain for decades: most notably the tricyclic antidepressants, amitriptyline, and nortriptyline. Once released from the presynaptic neuron, NA typically has a short-lived effect, as much of it is rapidly transported back into the nerve terminal. In blocking the reuptake of NA back into the presynaptic neurons, more neurotransmitter remains for a longer period of time and is therefore available for interaction with pre- and postsynaptic α₂-adrenergic receptors (AR). Tricyclic antidepressants and other NA reuptake inhibitors enhance the antinociceptive effect of opioids by increasing the availability of spinal NA. The α₂A-AR subtype is necessary for spinal adrenergic analgesia and synergy with opioids for most agonist combinations in both animal and humans (Chabot-Doré et al.; Neuropharmacology; 2015; 99; 285-300). A selective upregulation of spinal NET in a rat model of neuropathic pain with concurrent downregulation of serotonin transporters has been shown (Fairbanks et al.; Pharmacol. Ther.; 2009; 123; 224-238). Inhibitors of NA reuptake such as nisoxetine, nortriptyline and maprotiline and dual inhibitors of the noradrenaline and serotonin reuptake such as imipramine and milnacipran produce potent anti-nociceptive effects in the formalin model of tonic pain. Neuropathic pain resulting from the chronic constriction injury of the sciatic nerve was prevented by the dual uptake inhibitor, venlafaxine. In the spinal nerve ligation model, amitriptyline, a non-selective serotonin and noradrenaline reuptake blocker, the preferential noradrenaline reuptake inhibitor, desipramine and the selective serotonin and noradrenaline reuptake inhibitors, milnacipran and duloxetine, produce a decrease in pain sensitivity whereas the selective serotonin reuptake inhibitor, fluoxetine, is ineffective (Mochizucki, D.; Psychopharmacol.; 2004; Supplm. 1; S15-S19; Hartrick, C. T.; Expert Opin. Investig. Drugs; 2012; 21; 1827-1834). A number of nonselective investigational agents focused on noradrenergic mechanisms with the potential for additive or even synergistic interaction between multiple mechanisms of action are being developed.

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al., Nat. Biotechnol. 2009; 27: 659-666.).

In the case of chronic pain, which is a multifactorial disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactorial diseases such as pain (Gilron et al., Lancet Neurol. 2013 November; 12(11):1084-95.). In fact, positive synergistic interaction for several compounds, including analgesics, has been described (Schröder et al., J Pharmacol. Exp. Ther. 2011; 337:312-20. Erratum in: J Pharmacol. Exp. Ther. 2012; 342: 232; Zhang et al., Cell Death Dis. 2014; 5: e1138; Gilron et al., 2013, supra).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, Nat. Chem. Biol. 2008; 4:682-90).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008, supra).

There are two potentially important interactions between NET and α₂δ-1 inhibition: 1) synergism in analgesia, thus reducing the risk of specific side effects; and 2) inhibition of pain-related affective comorbidities such as anxiety and/or depressive like behaviors (Nicolson et al.; Harv. Rev. Psychiatry; 2009; 17; 407-420).
1) Preclinical research has demonstrated that gabapentinoids attenuated pain-related behaviors through supraspinal activation of the descending noradrenergic system (Tanabe et al.; J. Neurosci. Res.; 2008; Hayashida, K.; Eur. J. Pharmacol.; 2008; 598; 21-26). In consequence, the α₂δ-1-related analgesia mediated by NA-induced activation of spinal α2-adrenergic receptors can be potentiated by the inhibition of the NET. Some evidence from combination studies in preclinical models of neuropathic pain exist. Oral duloxetine with gabapentin was additive to reduce hypersensitivity induced by nerve injury in rats (Hayashida & Eisenach, 2008). The combination of gabapentin and nortriptyline was synergic in mice submitted to orofacial pain and to peripheral nerve injury model (Miranda, H. F. et al.; J. Orofac. Pain; 2013; 27; 361-366; Pharmacology; 2015; 95; 59-64).
2) Drug modulation of NET and α₂δ-1 has been shown to produce antidepressant and anti-anxiety effects respectively (Frampton, J. E.; CNS Drugs; 2014; 28; 835-854; Hajós, M. et al.; CNS Drug Rev.; 2004; 10; 23-44). In consequence, a dual drug that inhibited the NET and α₂δ-1 subunit of VGCC may also stabilize pain-related mood impairments by acting directly on both physical pain and the possible mood alterations.

Pain is multimodal in nature, since in nearly all pain states several mediators, signaling pathways and molecular mechanisms are implicated. Consequently, monomodal therapies fail to provide complete pain relief. Currently, combining existing therapies is a common clinical practice and many efforts are directed to assess the best combination of available drugs in clinical studies (Mao, J., Gold, M. S., Backonja, M.; 2011; J. Pain; 12; 157-166).

Accordingly, there is a need to find compounds that have an alternative or improved pharmacological activity in the treatment of pain, being both effective and showing the desired selectivity, and having good “drugability” properties, i.e. good pharmaceutical properties related to administration, distribution, metabolism and excretion.

In view of the existing results of the currently available therapies and clinical practices, the present invention offers a solution by combining in a single compound binding to two different targets relevant for the treatment of pain. This was mainly achieved by providing the compounds according to the invention that bind both to the noradrenaline transporter (NET) and to the α₂δ subunit, in particular the α₂δ-1 subunit, of the voltage-gated calcium channel.

### SUMMARY OF THE INVENTION

The authors of the present invention have found a series of compounds that show dual pharmacological activity towards both the α₂δ subunit, in particular the α₂δ-1 subunit, of the voltage-gated calcium channel, and the noradrenaline transporter (NET), resulting in an innovative, effective and alternative solution for the treatment of pain.

In this invention a family of structurally new compounds, encompassed by formula (I), which have a dual pharmacological activity towards both the α₂δ subunit, in particular the α₂δ-1 subunit, of the voltage-gated calcium channel, and the noradrenaline transporter (NET) has been identified, thus solving the above problem of identifying alternative or improved pain treatments and related disorders by offering such dual compounds.

The main object of the invention is directed to a compound having a dual activity binding to the α₂δ subunit, in particular the α₂δ-1 subunit, of the voltage-gated calcium channel and the noradrenaline transporter (NET) and the α₂δ-1 subunit of voltage-gated calcium channels, for use in the treatment of pain.

The invention is directed in a main aspect to a compound of formula (I),

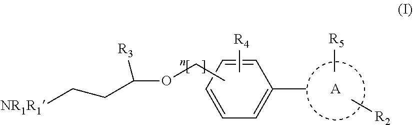

wherein R₁, R_{1′}, R₂, R₃, R₄, R₅, A and n are as defined below in the detailed description.

A further aspect of the invention refers to the processes for preparation of compounds of formula (I).

A still further aspect of the invention refers to the use of intermediate compounds for the preparation of a compound of formula (I).

It is also an aspect of the invention a pharmaceutical composition comprising a compound of formula (I).

Finally, it is an aspect of the invention a compound of formula (I) for use in therapy and more particularly for the treatment of pain and pain related conditions.

### DETAILED DESCRIPTION OF THE INVENTION

The invention is directed to a family of compounds which have a dual pharmacological activity towards both the α₂δ subunit, in particular the α₂δ-1 subunit, of the voltage-gated calcium channel and the NET receptor for use in the treatment of pain and related disorders.

In a first aspect, the present invention is directed to a compound of formula (I):

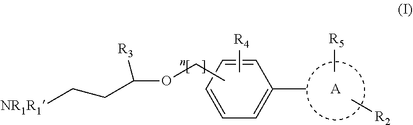

wherein
n is 0, 1, 2 or 3;
each R₁ and R_{1′} are independently selected from hydrogen, substituted or unsubstituted C₁₋₆ alkyl, substituted or unsubstituted C₂₋₆ alkenyl and substituted or unsubstituted C₂₋₆ alkynyl;
R₂ is selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl, haloalkyl, —OR₆, —SR₆ and —NR₆R_{6′};
wherein each R₆ and R_{6′} are independently selected from hydrogen, halogen, haloalkyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈ and —NR₈R_{8′};
each R₈ and R_{8′} are independently selected from hydrogen, unsubstituted C₁₋₆ alkyl, unsubstituted C₂₋₆ alkenyl, and unsubstituted C₂₋₆ alkynyl;
   R₃ is selected from substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
   R₄ is selected from hydrogen, —CN, halogen, substituted or unsubstituted C₁₋₆ alkyl, substituted or unsubstituted C₂₋₆ alkenyl, substituted or unsubstituted C₂₋₆ alkynyl, OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇;
wherein each R₇, R_{7′} and R_{7″} are independently selected from hydrogen, unsubstituted C₁₋₆ alkyl, unsubstituted C₂₋₆ alkenyl, and unsubstituted C₂₋₆ alkynyl;
   R₅ is selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl and haloalkyl;
   cycle A is an heterocyclyl;
wherein cycle A is linked to the phenyl moiety of the compound of formula (I) through a carbon atom; and
wherein cycle A and the group

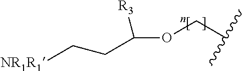

linked to the phenyl moiety of the compound of formula (I) stand in meta or para position to each other;
   wherein the compound of formula (I) is optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

The compounds of the invention represented by the above described formula (I) may include enantiomers depending on the presence of chiral centres or isomers depending on the presence of multiple bonds. The single isomers, enantiomers or diastereoisomers and mixtures thereof fall within the scope of the present invention.

In another embodiment, these compounds according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt or solvate thereof.

For the sake of clarity the expression “a compound according to formula (I), wherein R₁, R_{1′}, R₂, R₃, R₄, R₅, A and n are as defined herein in the detailed description” would (just like the expression “a compound of formula (I) as defined in any one of claims 1 to 11 found in the claims) refer to “a compound according to formula (I)”, wherein the definitions of the respective substituents R₁ etc. (also from the cited claims) are applied.

For clarity purposes, all groups and definitions described in the present description and referring to compounds of formula (I), also apply to all intermediate of synthesis.

In addition, and for clarity purposes, it should further be understood that naturally if n is 0, the oxygen is still present in formula (I).

For clarity purposes, reference is also made to the following statements below in the definitions of substitutions on alkyl etc. or aryl etc. that “wherein when different radicals R₁ to R_{8′} are present simultaneously in formula (I) they may be identical or different.

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2, C3- or C4-alkyl, C₁₋₅-alkyl represents C1-, C2-, C3-, C4-, or C5-alkyl and C₁₋₆-alkyl represents C1-, C2-, C3-, C4-, C5- or C6-alkyl. Examples of alkyl radicals include among others methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ and CH₂OH etc. Preferably alkyl is understood in the context of this invention C₁₋₆alkyl like methyl, ethyl, propyl, butyl, pentyl, or hexyl; and more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl).

Preferably in the context of this invention alkenyl is C₂₋₆-alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen, —OR′, —SR′, —SOR′, —SO₂R′, —OR′, —CN, —COR′, —COOR′, —NR′R′, —CONR′R′, haloalkyl, haloalkoxy or —OC₁₋₆ alkyl wherein each of the R′ groups is independently selected from the group consisting of hydrogen, OH, NO₂, NH₂, SH, ON, halogen, —COH, —COalkyl, —COOH, C1-C6 alkyl.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHC₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂Cl, —CH₂F, —CHCl₂, —CHF₂, and —CF₃.

In the context of this invention haloalkoxy is understood as meaning an —O-alkyl being substituted once or several times by a halogen (selected from F, Cl, Br, I). It encompasses e.g. —OCH₂Cl, —OCH₂F, —OCHCl₂, —OCHF₂, —OCCl₃, —OCF₃ and —OCH₂—CHCl₂. Preferably haloalkoxy is understood in the context of this invention as halogen-substituted —OC₁₋₄-alkyl representing halogen substituted C1-, C2-, C3- or C4-alkoxy. The halogen-substituted O-alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Preferred cycloalkyls are C₃₋₄-cycloalkyl representing C3- or C4-cycloalkyl, C₃₋₅-cycloalkyl representing C3-, C4- or C5-cycloalkyl, C₃₋₆-cycloalkyl representing C3-, C4-, C5- or C6-cycloalkyl, C₃₋₇-cycloalkyl representing C3-, C4-, C5-, C6- or C7-cycloalkyl, C₃₋₈-cycloalkyl representing C3-, C4-, C5-, C6-, C7- or C8-cycloalkyl, C₄₋₅-cycloalkyl representing C4- or C5-cycloalkyl, C₄₋₆-cycloalkyl representing C4-, C5- or C6-cycloalkyl, C₄₋₇-cycloalkyl representing C4-, C5-, C6- or C7-cycloalkyl, C₅₋₆-cycloalkyl representing C5- or C6-cycloalkyl and C₅₋₇-cycloalkyl representing C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantyl. Preferably in the context of this invention cycloalkyl is C₃₋₈cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 6 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphtyl or anthracenyl, more preferably the aryl is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms from the group consisting of nitrogen, oxygen and/or sulfur in the ring. A heterocyclic group can also be substituted once or several times.

Examples include non-aromatic heterocyclyls such as tetrahydropyrane, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic ring contains one or more heteroatoms from the group consisting of nitrogen, oxygen and/or sulfur in the ring; preferably is a 5 to 18 membered mono or polycyclic aromatic heterocyclic ring system of one or two rings of which at least one aromatic ring contains one or more heteroatoms from the group consisting of nitrogen, oxygen and/or sulfur in the ring, more preferably it is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably it is a 5 to 18 membered mono or polycyclic heterocyclic ring system of one or two rings of which one or both rings—with this one or two rings then not being aromatic—contain/s one or more heteroatoms from the group consisting of nitrogen, oxygen and/or sulfur in the ring, more preferably it is selected from oxazepam, pyrrolidine, piperidine, piperazine, tetrahydropyran, morpholine, indoline, oxopyrrolidine, benzodioxane, especially is benzodioxane, morpholine, tetrahydropyran, piperidine, oxopyrrolidine and pyrrolidine.

Preferably in the context of this invention heterocyclyl is defined as a 5 to 18 membered mono or polycyclic heterocyclic ring system of one or more saturated or unsaturated rings of which at least one ring contains one or more heteroatoms from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms from the group consisting of nitrogen, oxygen and/or sulfur in the ring.

Preferred examples of heterocyclyls include oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane and pyrrolidine. In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times.

Preferably alkylaryl is understood as meaning an aryl group (see above) being connected to another atom through 1 to 4 (—CH₂—) groups.

In the context of this invention alkylheterocyclyl is understood as meaning an heterocyclyl group (see above) being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups.

In the context of this invention alkylcycloalkyl is understood as meaning an cycloalkyl group (see above) being connected to another atom through a C₁₋₆-alkyl (see above) which may be branched or linear and is unsubstituted or substituted once or several times. Preferably alkylcycloalkyl is understood as meaning an cycloalkyl group (see above) being connected to another atom through 1 to 4 (—CH₂—) groups.

Preferably, the aryl is a monocyclic aryl. More preferably the aryl is a 6 or 7 membered monocyclic aryl. Even more preferably the aryl is a 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —R′, —OR′, —CN, —NO₂, —NR′R′, —COOR′, —NR′COR′, —CONR′R′, —NR′SO₂R′, ═O, —OCH₂CH₂OR′, —NR′CONR′R′, —SO₂NR′R′, —NR′SO₂NR′R′, haloalkyl, haloalkoxy, —SR′, —SOR′, —SO₂Rc or —C(CH₃)OR′; NR′R′, with R′ being either H or a saturated or unsaturated, linear or branched, substituted or unsubstituted C₁₋₆-alkyl; a saturated or unsaturated, linear or branched, substituted or unsubstituted —O—C1₋₆-alkyl (alkoxy); a saturated or unsaturated, linear or branched, substituted or unsubstituted —S—C₁₋₆-alkyl; a saturated or unsaturated, linear or branched, substituted or unsubstituted —CO—C₁₋₆-alkyl-group; a saturated or unsaturated, linear or branched, substituted or unsubstituted —CO—O—C₁₋₆-alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—), mesylate, nosylate or triflate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions. The definition particularly includes physiologically acceptable salts, this term must be understood as equivalent to “pharmacologically acceptable salts”.

The term “physiologically acceptable salt” means in the context of this invention any salt that is physiologically tolerated (most of the time meaning not being toxic-especially lacking toxicity caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this it is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

The compounds of the invention may be present in crystalline form or in the form of free compounds like a free base or acid.

Any compound that is a solvate of a compound according to the invention like a compound according to formula (I) defined above is understood to be also covered by the scope of the invention. Methods of solvation are generally known within the art. Suitable solvates are pharmaceutically acceptable solvates. The term “solvate” according to this invention is to be understood as meaning any form of the active compound according to the invention in which this compound has attached to it via non-covalent binding another molecule (most likely a polar solvent). Especially preferred examples include hydrates and alcoholates, like methanolates or ethanolates.

Any compound that is a prodrug of a compound according to the invention like a compound according to formula (I) defined above is understood to be also covered by the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a N-oxide of a compound according to the invention like a compound according to formula (I) defined above is understood to be also covered by the scope of the invention.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon or of a nitrogen by ¹⁵N-enriched nitrogen are within the scope of this invention.

The compounds of formula (I) as well as their salts or solvates are preferably in pharmaceutically acceptable or substantially pure form. By pharmaceutically acceptable pure form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts. This applies also to its solvates or prodrugs.

In a preferred embodiment the compound according to the invention of formula (I) is a compound wherein

n is 0, 1, or 2;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, the compound according to the invention of formula (I) is a compound wherein

n is 0 or 1, and preferably 1;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment, the compound according to the invention of formula (I) is a compound wherein

each R₁ and R_{1′} are independently selected from hydrogen and substituted or unsubstituted C₁₋₆ alkyl; preferably R₁ and R_{1′} are independently selected from hydrogen and methyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of formula (I) is a compound wherein

R₂ is selected from hydrogen, halogen, substituted or unsubstituted C₁₋₆ alkyl, substituted or unsubstituted heterocyclyl, haloalkyl, —OR₆, —SR₆, and —NR₆R₆′,
wherein each R₆ and R_{6′} are independently selected from hydrogen, halogen, substituted or unsubstituted C₁₋₆ alkyl, substituted or unsubstituted heterocyclyl, substituted or unsubstituted alkyheterocyclyl, OR₈, and —NR₈R₈′;
wherein each R₈ and R_{8′} are independently selected from hydrogen and unsubstituted C₁₋₆ alkyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compound according to the invention of formula (I) is a compound wherein R₂ is selected from hydrogen, halogen, substituted or unsubstituted C₁₋₆ alkyl, substituted or unsubstituted N-containing heterocyclyl, haloalkyl, —OR₆, —SR₆ and —NR₆R_{6′};
wherein each R₆ and R_{6′} are independently selected from hydrogen, halogen, substituted or unsubstituted C₁₋₆ alkyl, substituted or unsubstituted heterocyclyl, substituted or unsubstituted alkyheterocyclyl, OR₈, and —NR₈R₈′;
wherein each R₈ and R_{8′} are independently selected from hydrogen and unsubstituted C₁₋₆ alkyl;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compound according to the invention of formula (I) is a compound wherein R₂ is NR₆R_{6′} with R₆ and R_{6′} being independently selected from hydrogen, substituted or unsubstituted C₁₋₆ alkyl and substituted or unsubstituted heterocyclyl and, —NR₈R_{8′}, wherein each R₈ and R_{8′} are independently selected from hydrogen and unsubstituted C₁₋₆ alkyl;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a more preferred embodiment, R₆ is selected from hydrogen, substituted or unsubstituted C₁₋₆ alkyl.

In another preferred embodiment, R_{6′} is selected from hydrogen, substituted or unsubstituted C₁₋₆ alkyl and substituted or unsubstituted N-containing heterocyclyl.

In a further embodiment the compound according to the invention of formula (I) is a compound wherein

R₃ is selected from substituted or unsubstituted aryl, substituted or unsubstituted alkylaryl, substituted or unsubstituted cycloalkyl, substituted or unsubstituted alkylcycloalkyl, substituted or unsubstituted heterocyclyl and substituted or unsubstituted alkyheterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment the compound according to the invention of formula (I) is a compound wherein

R₃ is selected from substituted or unsubstituted aryl and substituted or unsubstituted heterocyclyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of formula (I) is a compound wherein

R₃ is selected from substituted or unsubstituted phenyl and substituted or unsubstituted S-containing heteroaryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compound according to the invention of formula (I) is a compound wherein:

R₃ is selected from phenyl and thiophene;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of formula (I) is a compound wherein:

R₄ is selected from hydrogen, —CN, halogen, substituted or unsubstituted C₁₋₆ alkyl, substituted or unsubstituted C₂₋₆ alkenyl, substituted or unsubstituted C₂₋₆ alkynyl, OR₇, —NO₂, —NR₇R_{7′″}, NR₇C(O)R_{7′}, —NR₇S(O)₂R_{7′}, —S(O)₂NR₇R_{7′}, —NR₇C(O)NR₇R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment, the compound according to the invention of formula (I) is a compound wherein:

R₄ is selected from hydrogen and —CN;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a further embodiment the compound according to the invention of formula (I) is a compound wherein:

R₅ is selected from hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, haloalkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof

In a preferred embodiment the compound according to the invention of formula (I) is a compound wherein:

R₅ is selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, of the compound according to the invention of formula (I) is a compound wherein: each R₆ and R_{6′} are independently selected from hydrogen, halogen, haloalkyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈ and —NR₈R_{8′};

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound according to the invention of formula (I) is a compound wherein:

each R₆ and R_{6′} are independently selected from hydrogen, halogen, substituted or unsubstituted C₁₋₆ alkyl and substituted or unsubstituted heterocyclyl; OR₈ and —NR₈R_{8′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment, the compound according to the invention of formula (I) is a compound wherein:

each R₆ and R_{6′} are independently selected from hydrogen, substituted or unsubstituted C₁₋₆ alkyl and substituted or unsubstituted alkylaryl, OR₈ and —NR₈R_{8′};
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another more preferred embodiment, the compound according to the invention of formula (I) is a compound wherein:

each R₆ is independently selected from hydrogen and substituted or unsubstituted C₁₋₆ alkyl;
each R_{6′} is independently selected from hydrogen, substituted or unsubstituted C₁₋₆ alkyl, substituted or unsubstituted N-containing heterocyclyl, OR₈ and —NR₈R₈; preferably R_{6′} is selected from hydrogen, substituted or unsubstituted C₁₋₆ alkyl and substituted or unsubstituted N-containing heterocyclyl; and
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another further embodiment, the compound according to the invention of formula (I) is a compound wherein:

each R₇ and R_{7′} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound according to the invention of formula (I) is a compound wherein:

each R₇ and R_{7′} are independently selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, the compound according to the invention of formula (I) is a compound wherein:

each R₈ and R_{8′} are independently selected from hydrogen, unsubstituted C₁₋₆ alkyl, unsubstituted C₂₋₆ alkenyl, and unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment the compound according to the invention of formula (I) is a compound wherein:

each R₈ and R_{8′} are independently selected from hydrogen and substituted or unsubstituted C₁₋₆ alkyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment the compound according the invention of formula (I) is a compound wherein:

cycle A is a heteroaryl; more preferably a N-containing heteroaryl.

In another preferred embodiment, the compound according to the invention of formula (I) is a compound wherein

Cycle A is linked to the phenyl moiety of the compound of formula (I) through carbon atom; wherein cycle A and the group

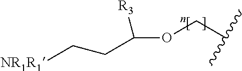

linked to the phenyl moiety of the compound of formula (I) stand in meta or para position to each other.

In another preferred embodiment, the compound according to the invention of formula (I) is a compound wherein Cycle A is linked to the phenyl moiety of formula (I) through carbon atom wherein cycle A and the group

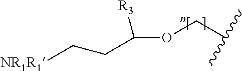

linked to the phenyl moiety of the compound of formula (I) stand in meta position to each other.

In another preferred embodiment, the compound according to the invention of formula (I) is a compound wherein Cycle A is linked to the phenyl moiety of formula (I) through carbon atom wherein cycle A and the group

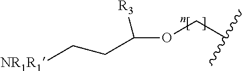

linked to the phenyl moiety of the compound of formula (I) stand in para position to each other.

In another preferred embodiment of the invention according to formula (I) the compound is a compound, wherein:

n is 0, 1, 2 or 3, and preferably n is 0 or 1;
and/or
each R₁ and R_{1′} are independently selected from hydrogen and substituted or unsubstituted C₁₋₆ alkyl; preferably R₁ and R_{1′} are independently selected from hydrogen and methyl;
and/or
R₂ is selected from hydrogen, substituted or unsubstituted C₁₋₆ alkyl, substituted or unsubstituted N-containing heterocyclyl, haloalkyl, —OR₆, —SR₆ and —NR₆R_{6′}; preferably R₂ is selected from hydrogen, methyl, substituted or unsubstituted piperazine, methyl piperazine, —CF₃, OR₆, —SR₆ and —NR₆R_{6′};
and/or
each R₆ and R_{6′} are independently selected from hydrogen, halogen, haloalkyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈ and —NR₈R_{8′}; preferably R₆ and R_{6′} are independently selected from hydrogen, halogen, substituted or unsubstituted C₁₋₆ alkyl and substituted or unsubstituted heterocyclyl, OR₈ and —NR₈R_{8′}; more preferably R₆ is selected from hydrogen, substituted or unsubstituted C₁₋₆ alkyl, substituted or unsubstituted N-containing heterocyclyl OR₈ and —NR₈R_{8′} and R_{6′} is selected from hydrogen and substituted or unsubstituted C₁₋₆ alkyl; and; even more preferably R₆ is methyl, ethyl, propyl, butyl, tertbutyl, pyrrolidine and hydroxyl pyrrolidine; and R_{6′} is selected from hydrogen and methyl;
and/or
R₈ and R_{8′} are independently selected from hydrogen, unsubstituted C₁₋₆ alkyl, unsubstituted C₂₋₆ alkenyl, and unsubstituted C₂₋₆ alkynyl; preferably R₈ and R_{8′} are independently selected from hydrogen and substituted or unsubstituted C₁₋₆ alkyl;
R₃ is selected from substituted or unsubstituted aryl and substituted or unsubstituted S-containing heteroaryl; preferably R₃ is selected from phenyl and thiophene;
and/or
R₄ is selected from hydrogen, —CN, halogen, substituted or unsubstituted C₁₋₆ alkyl, substituted or unsubstituted C₂₋₆ alkenyl, substituted or unsubstituted C₂₋₆ alkynyl, OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″} and C(CH₃)₂OR₇; preferably R₄ is selected from hydrogen and —CN;
and/or
R₇ and R_{7′} are independently selected from hydrogen, unsubstituted C₁₋₆ alkyl, unsubstituted C₂₋₆ alkenyl, and unsubstituted C₂₋₆ alkynyl; preferably R₇ and R_{7′} are independently selected from hydrogen and substituted or unsubstituted C₁₋₆ alkyl;
and/or
R₅ is selected from hydrogen and substituted or unsubstituted C₁₋₆ alkyl; preferably R₅ is selected from hydrogen and methyl;
and/or
wherein cycle A is a heteroaryl; and preferably a N-containing heteroaryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₁ and R_{1′} as defined in any of the embodiments of the present invention,
the C₁₋₆ alkyl is preferably selected from methyl, ethyl, propyl, butyl, pentyl, hexyl, isopropyl, or 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₂ as defined in any of the embodiments of the present invention,
the alkyl is preferably selected from C₁₋₆ alkyl; more preferably, the C₁₋₆ alkyl is selected from methyl, ethyl, propyl, butyl, pentyl, hexyl, isopropyl, or 2-methylpropyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphtyl, or anthracene; preferably is napthyl and phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline;
and/or
the haloalkyl is selected from —CH₂Cl, —CH₂F, —CHC₂, —CHF₂, —CCl₃, —CF₃ and —CH₂—CHCl₂; more preferably the haloalkyl is —CF₃
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₃ as defined in any of the embodiments of the present invention,
the cycloalkyl is C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl or cyclohexyl;
and/or
the aryl is selected from phenyl, naphtyl, or anthracene; preferably is phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline; preferably is thiophene;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₄ as defined in any of the embodiments of the present invention,
the C₁₋₆ alkyl is preferably selected from methyl, ethyl, propyl, butyl, pentyl, hexyl, isopropyl, and 2-methylpropyl, more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the haloalkyl is selected from —CH₂Cl, —CH₂F, —CHCl₂, —CHF₂, —CCl₃, —CF₃ and —CH₂—CHCl₂;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₅ as defined in any of the embodiments of the present invention,
the C₁₋₆ alkyl is preferably selected from methyl, ethyl, propyl, butyl, pentyl, hexyl, isopropyl, and 2-methylpropyl; more preferably the C₁₋₆ alkyl is methyl;
and/or
the C₂₋₆-alkenyl is preferably selected from ethylene, propylene, butylene, pentylene, hexylene, isopropylene and isobutylene;
and/or
the C₂₋₆-alkynyl is preferably selected from ethyne, propyne, butyne, pentyne, hexyne, isopropyne and isobutyne;
and/or
the cycloalkyl is C₃₋₈ cycloalkyl preferably selected from cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl, and cyclohexyl;
and/or
the aryl is selected from phenyl, naphtyl, and anthracene; preferably is napthyl and phenyl;
and/or
the heterocyclyl is a heterocyclic ring system of one or more saturated or unsaturated rings of which at least one ring contains one or more heteroatoms from the group consisting of nitrogen, oxygen and/or sulfur in the ring; preferably is a heterocyclic ring system of one or two saturated or unsaturated rings of which at least one ring contains one or more heteroatoms from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein in R₆ and R_{6′} as defined in any of the embodiments of the present invention,
each C₁₋₆ alkyl is preferably and independently selected from methyl, ethyl, propyl, butyl, pentyl, hexyl, isopropyl, and 2-methylpropyl; more preferably the C₁₋₆ alkyl is methyl, ethyl and tert-butyl;
and/or
each C₂₋₆-alkenyl is independently selected from ethylene, propylene, butylene, pentylene, hexylene, isopropylene and isobutylene;
and/or
each C₂₋₆-alkynyl is independently selected from ethyne, propyne, butyne, pentyne, hexyne, isopropyne and isobutyne;
and/or
each cycloalkyl is independently selected from C₃₋₈ cycloalkyl like cyclopropyl, cyclobutyl, cyclopentyl, cyclohexyl, cycloheptyl, or cyclooctyl; preferably is C₃₋₇ cycloalkyl like cyclopropyl, cyclobutyl, cyclopentyl, cyclohexyl, or cycloheptyl; more preferably from C₃₋₆ cycloalkyl like cyclopropyl, cyclobutyl, cyclopentyl and cyclohexyl;
and/or
each aryl is independently selected from phenyl, naphtyl, or anthracene; preferably is napthyl and phenyl; more preferably the aryl is phenyl;
and/or
each heterocyclyl is independently a heterocyclic ring system of one or more saturated or unsaturated rings of which at least one ring contains one or more heteroatoms selected from the group consisting of nitrogen, oxygen and/or sulfur in the ring; preferably is a heterocyclic ring system of one or two saturated or unsaturated rings of which at least one ring contains one or more heteroatoms selected from the group consisting of nitrogen, oxygen and/or sulfur in the ring, more preferably is selected from oxazepan, pyrrolidine, imidazole, oxadiazole, tetrazole, azetidine, pyridine, pyrimidine, piperidine, piperazine, benzofuran, benzimidazole, indazole, benzothiazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole, oxopyrrolidine, pyrimidine, benzodioxolane, benzodioxane, carbazole and quinazoline; more preferably is pyrrolidine and hydroxyl pyrrolidine;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention the compound according to formula (I) is a compound, wherein in R₇ and R_{7′} as defined in any of the embodiments of the present invention,
each C₁₋₆ alkyl is independently selected from methyl, ethyl, propyl, butyl, pentyl, hexyl, isopropyl, and 2-methylpropyl, more preferably the C₁₋₆ alkyl is selected from methyl, ethyl and propyl;
and/or
the C₂₋₆-alkenyl is independently selected from ethylene, propylene, butylene, pentylene, hexylene, isopropylene and isobutylene;
and/or
the C₂₋₆-alkynyl is independently selected from ethyne, propyne, butyne, pentyne, hexyne, isopropyne and isobutyne;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another preferred embodiment of the invention according to formula (I) the compound is a compound, wherein

n is 0, 1, 2 or 3; preferably n is 0 or 1;
and
each R₁ and R1′ are independently selected from hydrogen and substituted or unsubstituted C₁₋₆ alkyl; preferably R₁ and R1′ are independently selected from hydrogen and methyl;
and
R₂ is selected from hydrogen, substituted or unsubstituted C₁₋₆ alkyl, substituted or unsubstituted N-containing heterocyclyl, —OR₆, —SR₆ and —NR₆R_{6′}; preferably R₂ is selected from hydrogen, methyl, haloalkyl, substituted or unsubstituted piperazine, methyl piperazine, OR₆, —SR₆ and —NR₆R_{6′}
and
each R₆ and R_{6′} are independently selected from hydrogen, halogen, haloalkyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈ and —NR₈R_{8′}; preferably R₆ and R_{6′} are independently selected from hydrogen, halogen, substituted or unsubstituted C₁₋₆ alkyl and substituted or unsubstituted heterocyclyl, OR₈ and —NR₈R_{8′}; more preferably R₆ is selected from hydrogen, substituted or unsubstituted C₁₋₆ alkyl, substituted or unsubstituted N-containing heterocyclyl OR₈ and —NR₈R_{8′} and R_{6′} is selected from hydrogen and substituted or unsubstituted C₁₋₆ alkyl; and; even more preferably R₆ is methyl, ethyl, propyl, butyl, tertbutyl, pyrrolidine and hydroxyl pyrrolidine; and R_{6′} is selected from hydrogen and methyl;
and
each R₈ and R_{8′} are independently selected from hydrogen, unsubstituted C₁₋₆ alkyl, unsubstituted C₂₋₆ alkenyl, and unsubstituted C₂₋₆ alkynyl; preferably R₈ and R_{8′} are independently selected from hydrogen and substituted or unsubstituted C₁₋₆ alkyl;
and
R₃ is selected from substituted or unsubstituted aryl and substituted or unsubstituted S-containing heteroaryl; preferably R₃ is selected from phenyl and thiophene;
and
R₄ is selected from hydrogen, —CN, halogen, substituted or unsubstituted C₁₋₆ alkyl, substituted or unsubstituted C₂₋₆ alkenyl, substituted or unsubstituted C₂₋₆ alkynyl, OR₇, —NO₂, —NR₇R_{7′″}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR₇R_{7″}, and C(CH₃)₂OR₇; preferably R₄ is selected from hydrogen and —CN;
and
each R₇ and R_{7′} are independently selected from hydrogen, unsubstituted C₁₋₆ alkyl, unsubstituted C₂₋₆ alkenyl, and unsubstituted C₂₋₆ alkynyl; preferably R₇ and R_{7′} are independently selected from hydrogen and substituted or unsubstituted C₁₋₆ alkyl;
and
R₅ is selected from hydrogen and substituted or unsubstituted C₁₋₆ alkyl; preferably R₅ is selected from hydrogen and methyl;
and
wherein cycle A is linked to the phenyl moiety of general formula (I) through a carbon atom; preferably cycle A is a heteroaryl; more preferably a N-containing heteroaryl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an particular embodiment of the compound according to the invention of formula (I)

the halogen is fluorine, chlorine, iodine or bromine
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a most preferred embodiment of the compound according to the invention of formula (I)

the halogen is fluorine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In an embodiment of the compound according to the invention of formula (I), the haloalkyl is —CF₃;

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, the invention relates to a compound of formula (I) as described above, having one of the following formula (Ia) or (Ib):

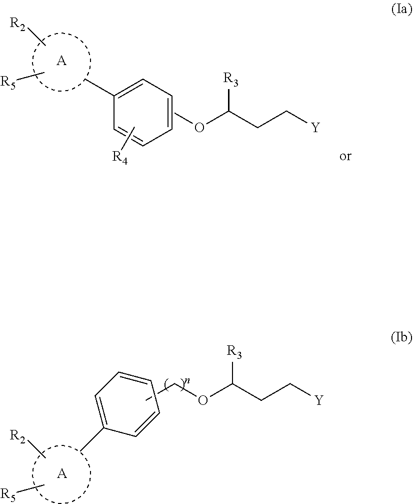

wherein in compound (Ib) n=1, 2 or 3 and wherein Y represents NR₁R_{1′} and R₁, R_{1′}, R₂, R₃, R₄, R₅, A and n are as defined above for a compound of formula (I).

In a preferred further embodiment, the compound of formula (I) is selected from:
[1] 3-(3-(2-Methoxypyrimidin-4-yl)phenoxy)-N-methyl-3-phenylpropan-1-amine,
[2] N-methyl-3-(3-(2-methylpyrimidin-4-yl)phenoxy)-3-phenylpropan-1-amine,
[3] N-methyl-3-(3-(6-methylpyridazin-3-yl)phenoxy)-3-phenylpropan-1-amine,
[4] N-methyl-3-phenyl-3-(3-(pyrimidin-4-yl)phenoxy)propan-1-amine,
[5] (S)—N-methyl-3-(3-(2-(methylthio)pyrimidin-4-yl)phenoxy)-3-phenylpropan-1-amine,
[6] (S)—N-methyl-3-(3-(2-methylpyrimidin-4-yl)phenoxy)-3-phenylpropan-1-amine,
[7] (R)—N-methyl-3-(3-(2-methylpyrimidin-4-yl)phenoxy)-3-phenylpropan-1-amine,
[8] (R)—N-methyl-3-(3-(2-(methylthio)pyrimidin-4-yl)phenoxy)-3-phenylpropan-1-amine,
[9] N-methyl-3-((3-(2-methylpyrimidin-4-yl)benzyl)oxy)-3-phenylpropan-1-amine,
[10] (S)-4-(3-(3-(methylamino)-1-phenylpropoxy)phenyl)pyrimidin-2-amine,
[11] (S)—N-(tert-butyl)-4-(3-(3-(methylamino)-1-phenylpropoxy)phenyl)pyrimidin-2-amine,
[12] N-methyl-3-phenyl-3-(3-(pyridin-2-yl)phenoxy)propan-1-amine,
[13] 3-((3-(2-methoxypyrimidin-4-yl)benzyl)oxy)-N-methyl-3-phenylpropan-1-amine,
[14] 3-(3-(6-methoxypyridin-2-yl)phenoxy)-N-methyl-3-phenylpropan-1-amine,
[15] N-methyl-3-phenyl-3-(3-(thiazol-2-yl)phenoxy)propan-1-amine,
[16] N-methyl-3-phenyl-3-(3-(thiophen-2-yl)phenoxy)propan-1-amine,
[17] N-Methyl-3-(3-(pyridin-3-yl)phenoxy)-3-(thiophen-2-yl)propan-1-amine,
[18] 3-(3-(2-methoxypyrimidin-5-yl)phenoxy)-N-methyl-3-(thiophen-2-yl)propan-1-amine,
[19] N-methyl-3-(3-(2-methylpyridin-4-yl)phenoxy)-3-(thiophen-2-yl)propan-1-amine,
[20] N-methyl-3-(3-(6-methylpyridin-3-yl)phenoxy)-3-(thiophen-2-yl)propan-1-amine,
[21] 3-(3-(2-methoxypyridin-4-yl)phenoxy)-N-methyl-3-(thiophen-2-yl)propan-1-amine,
[22] N-methyl-3-(thiophen-2-yl)-3-(3-(2-(trifluoromethyl)pyridin-4-yl)phenoxy)propan-1-amine,
[23] N-methyl-3-(3-(2-methylpyrimidin-5-yl)phenoxy)-3-(thiophen-2-yl)propan-1-amine,
[24] N-methyl-3-(3-(3-methylpyridin-4-yl)phenoxy)-3-(thiophen-2-yl)propan-1-amine,
[25] N-methyl-3-(3-(1-methyl-1,2,3,6-tetrahydropyridin-4-yl)phenoxy)-3-(thiophen-2-yl)propan-1-amine,
[26] 3-(3-(2,6-dimethylpyridin-4-yl)phenoxy)-N-methyl-3-(thiophen-2-yl)propan-1-amine,
[27] N-methyl-3-(3-(pyridin-4-yl)phenoxy)-3-(thiophen-2-yl)propan-1-amine,
[28] (S)—N-Methyl-3-phenyl-3-(3-(2-(piperazin-1-yl)pyrimidin-4-yl)phenoxy)propan-1-amine,
[29] (S)—N-ethyl-4-(3-(3-(methylamino)-1-phenylpropoxy)phenyl)pyrimidin-2-amine,
[30] (S)—N-methyl-4-(3-(3-(methylamino)-1-phenylpropoxy)phenyl)pyrimidin-2-amine,
[31] (S)—N-methyl-3-(3-(2-(4-methylpiperazin-1-yl)pyrimidin-4-yl)phenoxy)-3-phenylpropan-1-amine,
[32] (S)—N1-methyl-N2-(4-(3-(3-(methylamino)-1-phenylpropoxy)phenyl)pyrimidin-2-yl)ethane-1,2-diamine,
[33] 4-(3-((R)-3-(methylamino)-1-phenylpropoxy)phenyl)-N—((R)-pyrrolidin-3-yl)pyrimidin-2-amine,
[34] 4-(3-((R)-3-(methylamino)-1-phenylpropoxy)phenyl)-N—((S)-pyrrolidin-3-yl)pyrimidin-2-amine,
[35] (S)—N,N-dimethyl-4-(3-(3-(methylamino)-1-phenylpropoxy)phenyl)pyrimidin-2-amine,
[36] 4-(3-((S)-3-(methylamino)-1-phenylpropoxy)phenyl)-N—((R)-pyrrolidin-3-yl)pyrimidin-2-amine,
[37] 4-(3-((S)-3-(methylamino)-1-phenylpropoxy)phenyl)-N—((S)-pyrrolidin-3-yl)pyrimidin-2-amine,
[38] (3S,4S)-4-((4-(3-((S)-3-(methylamino)-1-phenylpropoxy)phenyl)pyrimidin-2-yl)amino)pyrrolidin-3-ol,
[39] (3S,4S)-4-((4-(3-((R)-3-(methylamino)-1-phenylpropoxy)phenyl)pyrimidin-2-yl)amino)pyrrolidin-3-ol,
[40] (3R,4R)-4-((4-(3-((S)-3-(methylamino)-1-phenylpropoxy)phenyl)pyrimidin-2-yl)amino)pyrrolidin-3-ol,
[41] (3R,4R)-4-((4-(4-((R)-3-(methylamino)-1-phenylpropoxy)phenyl)pyrimidin-2-yl)amino)pyrrolidin-3-ol,
[42] (3S,4S)-4-((4-(4-((R)-3-(methylamino)-1-phenylpropoxy)phenyl)pyrimidin-2-yl)amino)pyrrolidin-3-ol,
[43] (3S,4S)-4-((4-(4-((S)-3-(methylamino)-1-phenylpropoxy)phenyl)pyrimidin-2-yl)amino)pyrrolidin-3-ol,
[44] (3R,4R)-4-((4-(4-((S)-3-(methylamino)-1-phenylpropoxy)phenyl)pyrimidin-2-yl)amino)pyrrolidin-3-ol,
[45] 5-(2-(((3R,4R)-4-hydroxypyrrolidin-3-yl)amino)pyrimidin-4-yl)-2-((S)-3-(methylamino)-1-phenylpropoxy)benzonitrile,
[46] 5-(2-(((3R,4R)-4-hydroxypyrrolidin-3-yl)amino)pyrimidin-4-yl)-2-((R)-3-(methylamino)-1-phenylpropoxy)benzonitrile,
[47] 5-(2-(((3S,4S)-4-hydroxypyrrolidin-3-yl)amino)pyrimidin-4-yl)-2-((S)-3-(methylamino)-1-phenylpropoxy)benzonitrile,
[48] 5-(2-(((3S,4S)-4-hydroxypyrrolidin-3-yl)amino)pyrimidin-4-yl)-2-((R)-3-(methylamino)-1-phenylpropoxy)benzonitrile,
[49] N-Methyl-3-(3-(2-methylpyrimidin-4-yl)phenoxy)-3-(thiophen-2-yl)propan-1-amine,
[50] 3-(3-(2-methoxypyrimidin-4-yl)phenoxy)-N-methyl-3-(thiophen-2-yl)propan-1-amine,
[51] 4-(3-(3-(methylamino)-1-(thiophen-2-yl)propoxy)phenyl)pyrimidin-2-amine,
[52] N-methyl-3-(3-(2-(methylthio)pyrimidin-4-yl)phenoxy)-3-(thiophen-2-yl)propan-1-amine,
[53] (S)—N-Methyl-3-(3-(pyridin-4-yl)phenoxy)-3-(thiophen-2-yl)propan-1-amine,
[54] (S)—N-methyl-3-(3-(2-methylpyridin-4-yl)phenoxy)-3-(thiophen-2-yl)propan-1-amine,
[55] (R)—N-methyl-3-(3-(2-methylpyridin-4-yl)phenoxy)-3-(thiophen-2-yl)propan-1-amine,
[56] (S)—N-methyl-3-(3-(6-methylpyridin-3-yl)phenoxy)-3-(thiophen-2-yl)propan-1-amine,
[57] (R)—N-methyl-3-(3-(pyridin-4-yl)phenoxy)-3-(thiophen-2-yl)propan-1-amine,
[58] (R)—N-Methyl-3-(3-(1-methylpiperidin-4-yl)phenoxy)-3-(thiophen-2-yl)propan-1-amine,
[59] N-Methyl-3-(3-(pyridin-2-yl)phenoxy)-3-(thiophen-2-yl)propan-1-amine, and
[60] 4-(3-((R)-3-(Methylamino)-1-(thiophen-2-yl)propoxy)phenyl)-N—((S)-pyrrolidin-3-yl)pyrimidin-2-amine;
   optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

As mention above, this invention is aimed at providing a chemically related series of compounds which act as dual ligands of the α2δ subunit, particularly the α2δ-1 subunit, of the voltage-gated calcium channel and the NET receptor and especially compounds which have a binding expressed as Kᵢ responding to the following scales:

Kᵢ(NET) is preferably <1000 nM, more preferably <500 nM, and even more preferably <100 nM.

Kᵢ(α2δ1) is preferably <10000 nM, more preferably <5000 nM, and even more preferably <500 nM.

A preferred aspect of the invention is also a process for obtaining a compound of formula (I) as described above. Several procedures have been developed for obtaining all the compounds of the invention, and the procedures will be explained below in methods A and B.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. The compounds may be prepared in racemic form, or individual enantiomers may be prepared either by enantiospecific synthesis or by resolution.

One preferred pharmaceutically acceptable form of a compound of the invention is the crystalline form, including such form in pharmaceutical composition. In the case of salts and also solvates of the compounds of the invention the additional ionic and solvent moieties must also be non-toxic. The compounds of the invention may present different polymorphic forms, it is intended that the invention encompasses all such forms.

The compounds of formula (I) can be obtained by following the methods described below. As it will be obvious to one skilled in the art, the exact method used to prepare a given compound may vary depending on its chemical structure.

### Method A

Method A represents a first process for synthesizing compounds according to formula (I).

In this sense, in another aspect, the invention refers to a process for the preparation of a compound of formula (I)

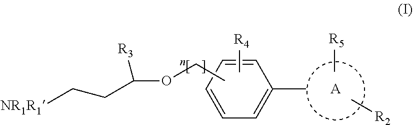

wherein Y represents NR₁R_{1′}, and R₁, R_{1′}, R₂, R₃, R₄, R₅, A and n are as defined above for a compound of formula (I); said process comprising:
treating a compound of formula (II)

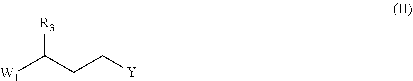

with a compound of formula (III):

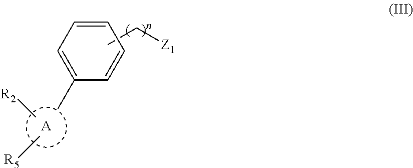

wherein
W₁ is OH or a leaving group such as halogen, mesylate, tosylate, nosylate or triflate, Z1 is OH or a leaving group such as halogen, mesylate, tosylate, nosylate or triflate, Y represents NR₁R_{1′}, NHR₁P or a leaving group such as halogen, mesylate, tosylate, nosylate or triflate and P represents a protecting group such as tert-butoxycarbonyl, 2-(trimethylsilyl)ethoxycarbonyl or benzyl, and R₁, R_{1′}, R₂, R₃, R₅, n, and A have the same meaning as indicated above for a compound of formula (I),
wherein
when W₁ is a leaving group such as halogen, mesylate, tosylate, nosylate or triflate, Z, is OH and n is 0.

Different reaction conditions are applied when when W₁ is OH, depending on whether n=0 (step 2a) or n=1-3 (step 2b).

The reactions steps are shown in Scheme 1 below in more detail:

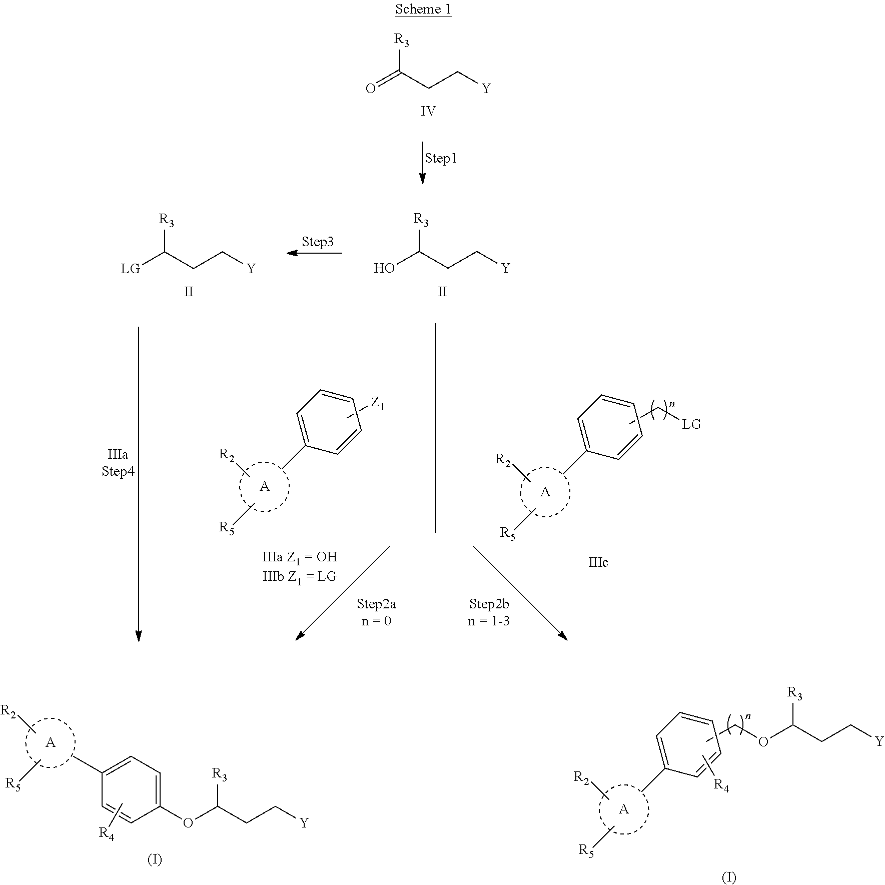

### Step 2a

A compound of formula (I) (n=0) can be prepared by reacting a compound of formula (II) with a suitable compound of formula (IIIa), particular case of compound of formula (III) wherein Z₁═OH or a compound of formula (IIIb), particular case of compound of formula (III) wherein Z₁=leaving group:
a) When a hydroxy compound of formula III is used, i.e. Z₁ is OH (IIIa) the reaction is carried out under conventional Mitsunobu conditions by treating an alcohol of formula II with a compound of formula IIIa in the presence of an azo compound such as 1,1′-(azodicarbonyl)dipiperidine (ADDP), diisopropylazodicarboxylate (DIAD) or diethyl azodicarboxylate (DEAD) and a phosphine such as tributylphosphine or triphenylphoshine. The Mitsunobu reaction is carried out in a suitable solvent, such as toluene or tetrahydrofuran; at a suitable temperature comprised between room temperature and the reflux temperature, preferably heating, or alternatively, the reactions can be carried out in a microwave reactor.
b) When a compound of formula IIIb, Z₁ being a leaving group, is used the reaction is carried out under conventional aromatic nucleophilic substitution conditions by treating an alcohol of formula II with a compound of formula IIIb wherein LG represents a leaving group (preferably fluoro), in the presence of a strong base such as sodium hydride. The reaction is carried out in a suitable solvent, such as a polar aprotic solvent, preferably dimethylformamide or dimethylacetamide; at a suitable temperature comprised between room temperature and the reflux temperature, preferably heating, or alternatively, the reactions can be carried out in a microwave reactor. Alternatively, when LG is triflate, bromo or iodo, the compound of formula IIIb can be introduced under cross-coupling conditions, using a Pd or Cu catalyst and a suitable ligand.

Alternatively, a compound of formula (I) n=0 can be prepared by reacting a compound of formula II (W₁=leaving group) with a phenolic compound of formula IIIa in the presence of a base such as potassium carbonate, in a suitable solvent, such as dimethylformamide, at a suitable temperature, such as reflux temperature. A compound of formula VIII can be prepared by converting the hydroxyl group of a compound of formula II to a leaving group, using a suitable reagent, such as thionyl chloride in a suitable solvent, such as dichloromethane, at a suitable temperature, such as room temperature.

Compounds of formula (II) can be obtained by the reduction of a keto compound of formula (IV)

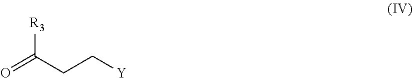

wherein Y represents NR₁R_{1′}, NHR₁P or a leaving group such as halogen, mesylate, tosylate, nosylate or triflate and P represents a protecting group such as tert-butoxycarbonyl, 2-(trimethylsilyl)ethoxycarbonyl or benzyl, and R₁, R_{1′} have the same meaning as defined before, following conventional procedures known in the art.

As a way of example, the reduction can be performed using a hydride source such as sodium borohydride in a suitable solvent such as methanol, ethanol or tetrahydrofuran or lithium aluminium hydride in a suitable solvent such as tetrahydrofuran or diethyl ether, at a suitable temperature, preferably comprised between 0° C. and room temperature.

Alternatively, the reduction can be carried out by hydrogenation under hydrogen atmosphere and metal catalysis, preferably by the use of palladium over charcoal or Nickel-Raney as catalysts, in a suitable solvent such as methanol, ethanol or ethyl acetate. In addition, the reduction can be performed under asymmetric conditions to render chiral compounds of formula II in enantiopure form. As a way of example, the chiral reduction can be performed using a hydride source such as borane-tetrahydrofuran complex or borane-dimethyl sulfide complex, in the presence of a Corey-Bakshi-Shibata oxazaborolidine catalyst, in a suitable solvent such as tetrahydrofuran or toluene, at a suitable temperature, preferably comprised between 0° C. and room temperature, following procedures already described in the art (i.e. WO2013/026455).

### Step 2b

A compound of formula (I) (n=1-3) can be prepared by reacting a compound of formula (II) with an alkylating agent of formula (IIIc), particular case of compound of formula (III) wherein Z₁=leaving group and n=1, 2 or 3 in the presence of a strong base such as sodium hydride or potassium tert-butoxide. The alkylation reaction is carried out in a suitable solvent, such as tetrahydrofuran or dimethylformamide, at a suitable temperature comprised between room temperature and the reflux temperature, preferably heating, or alternatively, the reactions can be carried out in a microwave reactor. Additionally, an activating agent such as sodium iodide or a phase transfer catalyst such as tetrabutylammonium iodide can be used.

For any of the steps explained above, the amino group NR₁R_{1′} can be incorporated at any step of the synthesis by reaction of a compound of formula II (W₁═OH), IV or (I) (n=0) and wherein Y is a leaving group such as chloro, bromo, iodo, mesylate, tosylate, nosylate or triflate with an amine of formula VII:

HNR₁R₁′ (VII)

wherein R₁ and R_{1′} have the same meaning as defined before. The alkylation reaction is carried out in a suitable solvent, such as ethanol, dimethylformamide, dimethylsulfoxide or acetonitrile, preferably ethanol; optionally in the presence of a base such as K₂CO₃ or triethylamine; at a suitable temperature comprised between room temperature and the reflux temperature, preferably heating, or alternatively, the reactions can be carried out in a microwave reactor. Additionally, an activating agent such as sodium iodide or potassium iodide can be used.

Additionally, it may be necessary to protect the amino group NR1R1′ or other reactive or labile groups present in the molecules with any suitable protecting group, such as for example Boc (tert-butoxycarbonyl), Teoc (2-(trimethylsilyl)ethoxycarbonyl) or benzyl for the protection of amino groups. The procedures for the introduction and removal of these protecting groups are well known in the art and can be found thoroughly described in the literature.

### Method B

Method B represents a second process for synthesizing compounds according to formula (I).

Accordingly, another aspect of the present invention relates to a process for the preparation of a compound of formula (I)

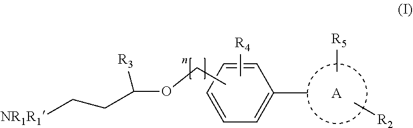

wherein Y represents NR₁R_{1′} and R₁, R_{1′}, R₂, R₃, R₄, R₅, A and n are as defined above for a compound of formula (I); said method comprising:
treating a compound of formula (XI)

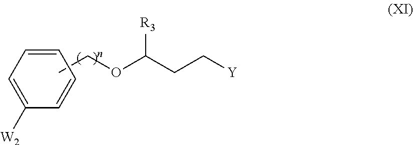

with a compound of formula (XIII)

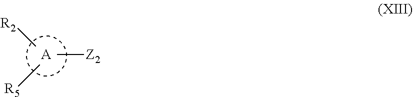

wherein
W₂ is BOM₂ or a leaving group such as halogen, mesylate, tosylate, nosylate or triflate; M represents hydrogen, alkyl or the two groups M can form a cycle together with the boron atom, Z₂ is OH or a leaving group such as halogen, mesylate, tosylate, nosylate or triflate, Y represents NR₁R_{1′}, NHR₁P or a leaving group such as halogen, mesylate, tosylate, nosylate or triflate and P represents a protecting group such as tert-butoxycarbonyl, 2-(trimethylsilyl)ethoxycarbonyl or benzyl, and R₁, R_{1′}, R₂, R₃, R₅ and A have the same meaning as indicated above for a compound of formula (I).

Alternatively, the compounds of formula (I) can be prepared by introducing the cyclic substituents AR₂R₅ along the synthesis, as depicted below in Scheme 2 in more detail:

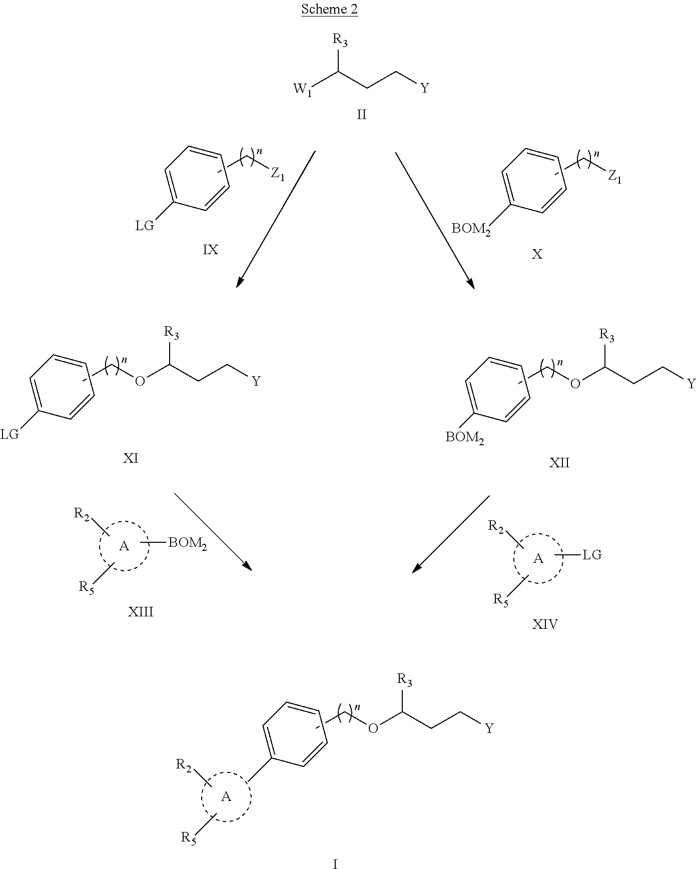

Thus, a compound of formula (II) wherein W₁═OH can be reacted with compounds of formula (IX) to give compounds of formula (XI) or with compounds of formula (X) to give compounds of formula (XII) using, depending on the nature of the reagents, the adequate conditions described above for Step 2. Alternatively, a compound of formula (II) wherein W₁=leaving group (LG) can be used as starting material to be reacted with compounds of formula (IX) (Z₁═OH) or (X) (Z₁═OH) in the presence of a base such as potassium carbonate, in a suitable solvent, such as dimethylformamide, at a suitable temperature, such as reflux temperature.

A compound of formula (XI) can be converted to a compound of formula (I) under cross-coupling conditions with a boronic derivative of formula (XIII), using a Pd or Cu catalyst and a suitable ligand. As a way of example suitable conditions include PdCl₂(dppf), in the presence of a base, such as sodium carbonate in a suitable solvent, such as water, at a suitable temperature, such as reflux temperature. Alternatively, a boronic derivative of formula (XII) can be converted to a compound of formula (I) by cross-coupling reaction with a compound of formula (XIV) under the same conditions.

The compounds of formula (II) are commercially available or can be obtained as described above from the corresponding compounds of formula (IV). The compounds of formula (IV) are commercially available or can be synthesized following procedures described in the literature. As a way of example, one route of synthesis involves the Friedel-Crafts acylation of an heteroaryl compound of formula R₃H with an acid halide in the presence of a Lewis acid such as aluminum trichloride. The reaction is carried out in a suitable solvent, such as dichloromethane or dichloroethane; at a suitable temperature comprised between 0° C. and the reflux temperature.

The compounds of formula III, VII, IX, X, XII, XIV are commercially available or can be prepared by conventional methods described in the bibliography.

Accordingly, another aspect of the present invention relates to the use of a compound according to formula II, III, IX, X, XI, XII, XIII or XIV

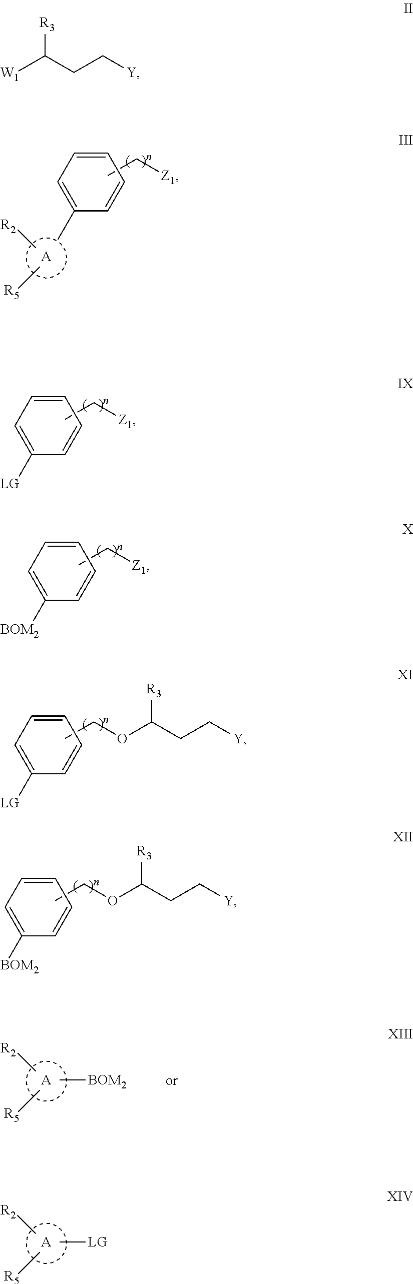

wherein
W₁ is OH or a leaving group such as halogen, mesylate, tosylate, nosylate or triflate, Z₁ is OH or a leaving group such as halogen, mesylate, tosylate, nosylate or triflate, Y represents NR₁R_{1′}, NHR₁P or a leaving group such as halogen, mesylate, tosylate, nosylate or triflate and P represents a protecting group such as tert-butoxycarbonyl, 2-(trimethylsilyl)ethoxycarbonyl or benzyl, M represents hydrogen, alkyl or the two groups M can form a cycle together with the boron atom, and R₁, R_{1′}, R₂, R₃, R₅, n, and A have the same meaning as indicated above for the preparation of a compound of formula (I).

Moreover, certain compounds of the present invention can also be obtained starting from other compounds of formula (I) by appropriate conversion reactions of functional groups, in one or several steps, using well-known reactions in organic chemistry under standard experimental conditions. As a way of example, some of these conversions include the reduction of a double bond from a tetrahydropyridine to a piperidine derivative or the reductive amination of an amino group with an aldehyde or ketone, or alternatively the reaction of an amino group with an alkylating agent, to prepare a further substituted amino group.

In addition, a compound of formula (I) that shows chirality can also be obtained by resolution of a racemic compound of formula (I) either by chiral preparative HPLC or by crystallization of a diastereomeric salt or co-crystal. Alternatively, the resolution step can be carried out at a previous stage, using any suitable intermediate.

Another aspect of the invention refers to a pharmaceutical composition which comprises a compound according to the invention as described above according to formula (I) or a pharmaceutically acceptable salt thereof, prodrug, solvate or stereoisomer thereof, and a pharmaceutically acceptable carrier, adjuvant or vehicle. The present invention thus provides pharmaceutical compositions comprising a compound of this invention, or a pharmaceutically acceptable salt, prodrug, solvate or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

The compounds and compositions of this invention may be used with other drugs to provide a combination therapy. The other drugs may form part of the same composition, or be provided as a separate composition for administration at the same time or at different time.

Another aspect of the invention refers to a compound of formula (I) as described above, or a pharmaceutical acceptable salt or isomer thereof for use in therapy.

Another aspect of the invention refers to a compound of formula I, or a pharmaceutically acceptable salt or isomer thereof, for use in the treatment or prophylaxis of pain. Preferably, the pain is medium to severe pain, visceral pain, chronic pain, cancer pain, migraine, inflammatory pain, acute pain or neuropathic pain, allodynia or hyperalgesia. This may include mechanical allodynia or thermal hyperalgesia.

Another aspect of the invention refers to the use of a compound of the invention in the manufacture of a medicament for the treatment or prophylaxis of pain. In a preferred embodiment the pain is selected from medium to severe pain, visceral pain, chronic pain, cancer pain, migraine, inflammatory pain, acute pain or neuropathic pain, allodynia or hyperalgesia, also preferably including mechanical allodynia or thermal hyperalgesia.

Another aspect of this invention relates to a method of treating or preventing pain which method comprises administering to a patient in need of such a treatment or prevention a therapeutically effective amount of a compound as above defined or a pharmaceutical composition thereof. Among the pain syndromes that can be treated or prevented are medium to severe pain, visceral pain, chronic pain, cancer pain, migraine, inflammatory pain, acute pain or neuropathic pain, allodynia or hyperalgesia, whereas this could also include mechanical allodynia or thermal hyperalgesia.

The present invention is illustrated below with the aid of examples. These illustrations are given solely by way of example and do not limit the general spirit of the present invention.

### EXAMPLES

The following abbreviations are used in the examples:

Boc: tert-butoxycarbonyl
BuLi: butyl lithium
Conc: concentrated

### CH: Cyclohexane

DCM: dichloromethane
DEA: diethylamine
DIAD: diisopropyl azodicarboxylate

### DMA: N,N-dimethylacetamide

Eq: equivalent/s
Et₂O: diethyl ether
EtOAc: ethyl acetate
EtOH: ethanol
EX: example
h: hour/s
HPLC: high performance liquid chromatography
2-Me-CBS-oxazaborolidine: 5,5-diphenyl-2-methyl-3,4-propano-1,3,2-oxazaborolidine (Corey-Bakshi-Shibata oxazaborolidine catalyst)
MeOH: methanol
MS: mass spectrometry
Min: minutes
PPh₃: triphenylphosphine
Quant: quantitative
Ret: retention
r.t.: room temperature
Sat: saturated
TEA: triethylamine
TFA: trifluoroacetic acid
THF: tetrahydrofuran
Wt: weight

The following methods were used to determine the HPLC-MS spectra:

### Method A

Column Aquity UPLC BEH C18 2.1×50 mm, 1.7 μm, flow rate 0.61 mL/min; temperature 35° C.; A: NH₄HCO₃ 10 mM, B: ACN; gradient 0.3 min 98% A, 98% to 0% A in 2.7 min; isocratic 2 min 0% A.

### Method B

Column Aquity UPLC BEH C18 2.1×50 mm, 1.7 μm, flow rate 0.61 mL/min; temperature 35° C.; A: NH₄HCO₃ 10 mM, B: ACN; gradient 0.3 min in 98% A, 98% A to 5% A in 2.52 min, isocratic 1.02 min in 5% A.

### Method C

Column Aquity UPLC BEH C18 2.1×50 mm, 1.7 μm; flow rate 0.60 mL/min; A: NH₄HCO₃ 10 mM; B: ACN; Gradient: 0.3 min 90% A, 90% to 5% A in 2.7 min, isocratic 5% A 0.7 min.

### Method D

Column Aquity UPLC BEH C18 2.1×50 mm, 1.7 μm; flow rate 0.60 mL/min; A: NH₄HCO₃ 10 mM; B: ACN; Gradient: 0.3 min 90% A, 90% to 5% A in 2.7 min, isocratic 5% A 0.7 min.

### Method E

Column Aquity UPLC BEH C18 2.1×50 mm, 1.7 μm; flow rate 0.60 mL/min; A: H₂O+0.05% TFA; B: ACN+0.04% TFA; Gradient: 0.3 min 90% A, 90% to 5% A in 2.7 min, isocratic 5% A 0.7 min.

### Method F

Column Aquity UPLC BEH C18 2.1×50 mm, 1.7 μm; flow rate 0.60 mL/min; A: NH₄HCO₃ 10 mM pH 10.6; B: ACN; Gradient: 0.3 min 90% A, 90% to 5% A in 2.7 min, isocratic 5% A 0.7 min.

### Synthesis of Intermediates

### Intermediate 1: tert-Butyl methyl(3-phenyl-3-(3-(4,4,5,5-tetramethyl-1,3,2-dioxaborolan-2-yl)phenoxy)propyl)carbamate

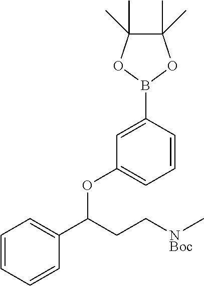 [SMILES: CN(CCC(Oc1cccc(B2OC(C)(C)C(C)(C)O2)c1)c1ccccc1)C(=O)OC(C)(C)C]

### Step 1. 3-Chloro-N-methyl-3-phenylpropan-1-amine hydrochloride

To a solution of 3-(methylamino)-1-phenylpropan-1-ol (5 g, 30.3 mmol) in DCM (20 mL) at 0° C., thionyl chloride (2.46 mL, 33.3 mmol) in DCM (7 mL) was added. The solution was allowed to reach r.t. and stirred for 2 h. The solvent was removed under reduced pressure to give the title compound as white solid (6.67 g, quant. yield).

### Step 2. tert-Butyl (3-chloro-3-phenylpropyl)(methyl)carbamate

To a NaOH solution (2 N, 32 mL, 63.6 mmol), a solution of the compound obtained in step 1 (7 g, 31.8 mmol) and Boc₂O (7.63 g, 35 mmol) in tBuOH (25 mL) was added. The reaction mixture was stirred at r.t for 10 minutes. Brine was added and the phases were separated. The aqueous phase was extracted with DCM and the combined organic fractions were dried over Mg₂SO₄, filtered and concentrated under reduced pressure to give the title compound (6.92 g, 24.4 mmol).

### Step 3. Title Compound

To a solution of the compound obtained in step 2 (775 mg, 3.52 mmol) in dry DMF (15 mL), K₂CO₃ (1.46 g, 10.6 mmol) and tert-butyl 3-chloro-3-phenylpropyl(methyl)carbamate (1 g, 3.52 mmol) were added. The reaction mixture was heated at 105° C. for 16 h. A few drops of water were added to the mixture and the volatiles evaporated. The residue was taken up in EtOAc and washed with water. The organic phase was dried over Mg₂SO₄, filtered and concentrated under reduced pressure to give the crude product (1.39 g) which was used in the next step without further purification.

HPLC ret time (method A): 2.86 min; ESI+MS: m/z 468.4 [M+H]⁺.

This method was used for the preparation of Intermediate 2 using suitable starting materials

| | | Chemical |
|---|---|---|
| INT | Structure | name |
| 2 | 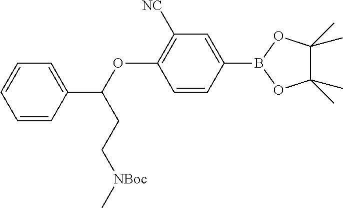 [SMILES: CN(CCC(Oc1ccc(B2OC(C)(C)C(C)(C)O2)cc1C#N)c1ccccc1)C(=O)OC(C)(C)C] | tert-butyl (3-(2-cyano-4- (4,4,5,5- tetramethyl- 1,3,2- dioxaborolan- 2-yl) phenoxy)-3- phenylpropyl) (methyl) c [symbol] |

[symbol]

indicates data missing or illegible when filed

### Intermediate 3: (R)-tert-Butyl methyl(3-phenyl-3-(3-(4,4,5,5-tetramethyl-1,3,2-dioxaborolan-2-yl)phenoxy)propyl)carbamate

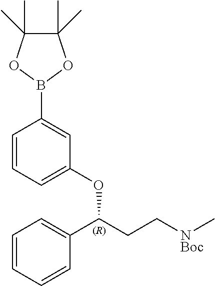

### Step 1. (R)-2-(3-(3-Chloro-1-phenylpropoxy)phenyl)-4,4,5,5-tetramethyl-1,3,2-dioxaborolane

To a solution of (S)-3-chloro-1-phenylpropan-1-ol (1 g, 5.86 mmol) in dry THF (10 mL), 3-(4,4,5,5-tetramethyl-1,3,2-dioxaborolan-2-yl)phenol (1.94 g, 8.79 mmol) and triphenylphosphine (2.31 g, 8.79 mmol) were added. The reaction solution was cooled to 0° C. and DIAD (1.78 g, 8.79 mmol) was added dropwise. After the addition was complete, the reaction mixture was stirred at r.t. for 3 days. The solvent was removed under reduced pressure and the residue purified by combiflash chromatography (neutral alumina, CH/EtOAc up to 30%) to give the title compound (623 mg, 29% yield).

### Step 2. (R)—N-Methyl-3-phenyl-3-(3-(4,4,5,5-tetramethyl-1,3,2-dioxaborolan-2-yl)phenoxy)propan-1-amine

To a solution of the compound obtained in step 1 (2.17 g, 5.81 mmol) in abs. EtOH (10 mL), methylamine (40% solution in water, 15 mL, 173 mmol) was added and the reaction mixture was heated in a sealed tube at 130° C. for 1 h. Water was added to the mixture and EtOH was removed under reduced pressure. The aqueous mixture was extracted with DCM and the combined organic fractions were dried over Mg₂SO₄, filtered and concentrated under reduced pressure to give the title compound (2.13 g, quant. yield).

### Step 3. Title Compound

To a solution of the compound obtained in step 2 (614 mg, 1.67 mmol) in dry DCM (15 mL), di-tert-butyl-dicarbonate (401 mg, 1.84 mmol) was added at 0° C. The reaction mixture allowed to reach to reach r.t. and was stirred for 2 h. To the reaction mixture, water was added and the aqueous phase was extracted with DCM. The combined organic fractions were dried over Mg₂SO₄ and concentrated under reduced pressure to give the title compound (761 mg, 97%).

HPLC ret time (method B): 2.82 min; ESI+MS: m/z 468.20 [M+H]⁺.

This method was used for the preparation of Intermediates 4-6, using suitable starting materials:

| INT | Structure | Chemical name |
|---|---|---|
| 4 | 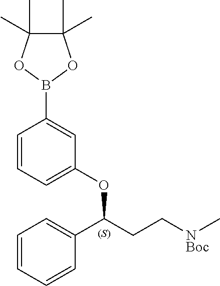 | (S)-tert-butyl methyl(3- phenyl-3-(3-(4,4,5,5- tetramethyl-1,3,2- dioxaborolan-2- yl)phenoxy)propyl) carba [symbol] |
| 5 | 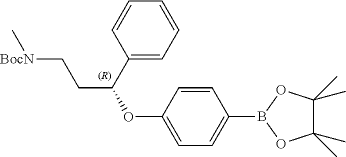 | (R)-tert-butyl methyl(3- phenyl-3-(4-(4,4,5,5- tetramethy1-1,3,2- dioxaborolan-2- [symbol] |
| 6 | 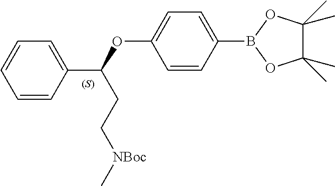 | (S)-tert-butyl methyl(3- phenyl-3-(4-(4,4,5,5- tetramethyl-1,3,2- dioxaborolan-2- [symbol] |

[symbol]

indicates data missing or illegible when filed

### Intermediate 7: tert-Butyl methyl(3-phenyl-3-((3-(4,4,5,5-tetramethyl-1,3,2-dioxaborolan-2-yl)benzyl)oxy)propyl)carbamate

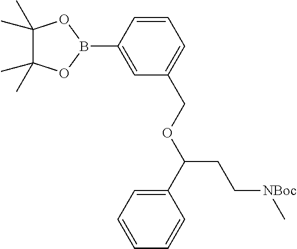

### Step 1: tert-Butyl (3-hydroxy-3-phenylpropyl)(methyl)carbamate

To a solution of 3-(methylamino)-1-phenylpropan-1-ol (2 g, 12.1 mmol) in dry DCM (12 mL), di-tert-butyl dicarbonate (2.91 g, 13.3 mmol) was added at 0° C. and the reaction mixture stirred for 2 h at r.t. Water was added and the aqueous phase was extracted with DCM. The combined organic fractions were washed with sat. NaHCO₃ solution, brine and dried over Mg₂SO₄. After filtration the solvent was removed under reduced pressure to afford the title compound (3.2 g, quant. yield) as oil.

### Step 2. Title Compound

To a solution of the compound obtained in step 1 (50 mg, 0.188 mmol) in DMF (3 mL) cooled at 0° C., NaH (60% suspension in mineral oil, 23 mg, 0.565 mmol) was added and the mixture was stirred at r.t. for 30 min. The reaction mixture was cooled again at 0° C. and TBAI (7 mg, 0.02 mmol) and a solution of 2-(3-(bromomethyl)phenyl)-4,4,5,5-tetramethyl-1,3,2-dioxaborolane (112 mg, 0.377 mmol) in DMF (2 mL) were added. The reaction mixture was gradually allowed to reach r.t. and stirred for 16 h. The solvent was removed under reduced pressure, the residue was partitioned between water and EtOAc and the aqueous phase was extracted with EtOAc. The combined organic fractions were dried over Na₂SO₄, filtered and the solvent was removed under reduced pressure to afford the title compound as oil (114 mg), which was used in the next step without further purification.

HPLC ret time (method A): 2.94 min; ESI+MS: m/z 482.33 [M+H]⁺.

### Intermediate 8: 2-(Trimethylsilyl)ethyl methyl(3-(3-(4,4,5,5-tetramethyl-1,3,2-dioxaborolan-2-yl)phenoxy)-3-(thiophen-2-yl)propyl)carbamate

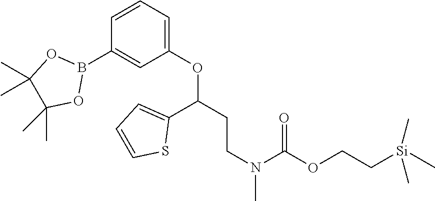

### Step 1: 3-Chloro-1-(thiophen-2-yl)propan-1-one

3-Chloropropanoyl chloride (12.4 ml, 130 mmol) in dry DCM (50 mL) was added dropwise to a stirred suspension of aluminium chloride (18.8 g, 141 mmol) in dry DCM (100 mL) at −5° C. The resulting suspension was allowed to stir at −5° C. for 10 min before a solution of thiophene (10 g, 118 mmol) in dry DCM (50 mL) was added dropwise. The resulting orange solution was stirred at −5° C. for 1 h and quenched by adding crushed ice (200 g). The organic phase was separated, dried over Mg₂SO₄ and filtered. Removal of the solvent under reduced pressure gave the title compound as oil (16 g, 77% yield).

### Step 2: 3-Chloro-1-(thiophen-2-yl)propan-1-ol

To a solution of the compound obtained in step 1 (5 g, 28.6 mmol) in MeOH (100 mL) NaBH₄ (2.71 g, 71.6 mmol) was added at 0° C. and the mixture was stirred at room temperature for 16 h. The reaction was quenched with some drops of water and the solvent was removed under reduced pressure. The residue was dissolved in EtOAc and washed with water and brine. The organic phase was dried over Na₂SO₄, filtered and concentrated under reduced pressure to afford the title compound as oil (4.77 g, 94% yield).

### Step 3: 2-(3-(3-Chloro-1-(thiophen-2-yl)propoxy)phenyl)-4,4,5,5-tetramethyl-1,3,2-dioxaborolane

To a solution of the compound obtained in step 2 (4.77 g, 27 mmol), 3-(4,4,5,5-tetramethyl-1,3,2-dioxaborolan-2-yl)phenol (8.91 g, 40.5 mmol) and triphenylphosphine (10.6 g, 40.5 mmol) in dry THF (25 mL) were added. The reaction mixture was cooled to 0° C. and DIAD (8.19 g, 40.5 mmol) was added dropwise. The reaction was allowed to reach r.t. and stirred for 3 days. The solvent was removed under reduced pressure.

The residue was suspended in diethyl ether and filtered. The filtrate was concentrated under reduced pressure and the remaining residue purified by combiflash chromatography (Neutral alumina, CH/EtOAc up to 40%) followed by repurification by combiflash (SiO₂, CH/EtOAc up to 40%) to give the title compound.

### Step 4: N-Methyl-3-(3-(4,4,5,5-tetramethyl-1,3,2-dioxaborolan-2-yl)phenoxy)-3-(thiophen-2-yl)propan-1-amine

To a solution of the compound obtained in step 3 (1 g, 2.64 mmol) in abs. EtOH (15 mL), methylamine (40% solution in water, 10 mL, 115 mmol) was added and the reaction mixture heated in a sealed tube at 130° C. for 1 h. The volatiles were removed under reduced pressure. Water and DCM were added to the residue and the phases separated. The organic phase was dried over MgSO₄, filtered and concentrated under reduced pressure to give the title compound (989 mg, quant. yield) as oil.

### Step 5: Title Compound

To a solution of 2-(trimethylsilyl)ethanol (931 mg, 7.88 mmol) and K₂CO₃ (1.45 g, 10.5 mmol) in toluene (10 mL) at 0° C., triphosgene (811 mg, 2.63 mmol) in toluene (2 mL) was added dropwise. The reaction mixture was stirred for 1 h at r.t. and cooled back to 0° C. To this mixture, a solution of the compound obtained in step 4 in toluene (4 mL) was added and the resulting reaction mixture was allowed to stir at r.t. for 3 h. The reaction mixture was poured into sat. aqueous NaHCO₃ solution and extracted with EtOAc. The combined organic fractions were dried over MgSO₄, filtered and concentrated under reduced pressure to give the title compound (1.66 g) which was used in the next step without further purification.

HPLC ret time (method A): 3.01 min; ESI−MS: m/z 534.2 [M−H+H₂O]⁻.

### Intermediate 9: 2-(Trimethylsilyl)ethyl (3-(3-iodophenoxy)-3-(thiophen-2-yl)propyl)(methyl)carbamate

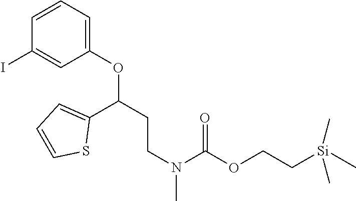 [SMILES: CN(CCC(Oc1cccc(I)c1)c1cccs1)C(=O)OCC[Si](C)(C)C]

### Step 1: 2-(3-Chloro-1-(3-iodophenoxy)propyl)thiophene

To a mixture of 3-chloro-1-(thiophen-2-yl)propan-1-ol (prepared as described in intermediate 8, 1 g, 5.66 mmol), 3-iodophenol (1.37 g, 6.23 mmol) and triphenylphosphine (1.63 g, 6.23 mmol) in dry THF (30 mL) DIAD (1.28 g, 6.34 mmol) was added dropwise at 0° C. The reaction mixture was stirred at r.t. for 3 days. The solvent was removed under reduced pressure and the residue suspended in EtOAc and filtered. The filtrate was concentrated under reduced pressure and the residue purified by combiflash chromatography (SiO₂, CH/EtOAc up to 40%) to give the title compound (610 mg, 28% yield) as oil.

### Step 2: 3-(3-Iodophenoxy)-N-methyl-3-(thiophen-2-yl)propan-1-amine

To a solution of the compound obtained in step 1 in abs. EtOH (2.5 mL) methylamine (40% solution in water, 5 mL, 115 mmol) was added and the resulting solution heated in a sealed tube at 100° C. for 1 h. The reaction was quenched with water and EtOH was removed under reduced pressure. The aqueous mixture was extracted twice with DCM. The combined organic fractions were dried over MgSO₄, filtered and concentrated under reduced pressure to give the title compound (501 mg, quant. yield).

### Step 3: Title Compound

To a solution of the compound obtained in step 2 (500 mg, 1.34 mmol) in DCM (15 mL), DIPEA (233 mL, 1.34 mmol) and 4-nitrophenyl (2-(trimethylsilyl)ethyl) carbonate (380 mg, 1.34 mmol) in DCM (5 mL) were added. The reaction mixture was stirred at r.t. for 16 h. Sat. aqueous NaHCO₃ solution was added to the mixture and the product extracted with DCM. The combined organic fractions were washed with 10% NaOH (3×) and dried over MgSO₄. After filtration, the solvent was removed under reduced pressure to give a residue which was purified by combiflash chromatography (SiO₂, CH/EtOAc up to 100%) to give the title compound (452 mg, 65% yield) as oil.

HPLC ret time (method A): 2.97 min; ESI−MS: m/z 516.2 [M−H]⁻.

### Intermediate 10: (S)-2-(Trimethylsilyl)ethyl (3-(3-bromophenoxy)-3-(thiophen-2-yl)propyl)(methyl)carbamate

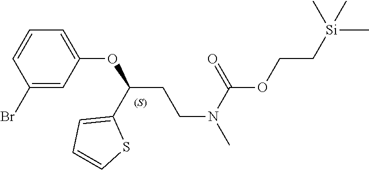

### Step 1. (S)-3-(3-Bromophenoxy)-N-methyl-3-(thiophen-2-yl)propan-1-amine

To a solution of (S)-3-(methylamino)-1-(thiophen-2-yl)propan-1-ol (215 mg, 1.3 mmol) in DMA (8 mL), NaH (60% suspension in mineral oil, 100 mg, 2.51 mmol) was added and the solution was stirred at r.t. for 30 min. Then, 1-bromo-3-fluoro benzene (0.28 mL, 2.5 mmol) was added and the mixture was heated at 90° C. for 3 h. Water was added and the product extracted with Et₂O and AcOEt. The combined organic fractions were dried over Na₂SO₄, filtered and the solvent was removed under reduced pressure affording the title compound (370 mg, 90% yield) as oil.

### Step 2. Title Compound

To a solution of the compound obtained in step 1 (369 mg, 1.13 mmol) in DCM (10 mL), DIPEA (197 μL, 1.13 mmol) was added, followed by the dropwise addition of 4-nitrophenyl (2-(trimethylsilyl)ethyl) carbonate (320 mg, 1.31 mmol) in DCM (2 mL). The reaction mixture was stirred for 16 h. Sat. aqueous NaHCO₃ solution was added to the mixture and the product extracted with DCM. The combined organic fractions were washed with 10% NaOH, dried over MgSO₄, filtered and the solvent removed under reduced pressure to give the title compound (481 mg, 90% yield.

HPLC ret time (method A): 2.89 min; ESI−MS: m/z 468.1 [M+H]⁻.

### Intermediate 11: (R)-2-(Trimethylsilyl)ethyl (3-(3-bromophenoxy)-3-(thiophen-2-yl)propyl)(methyl)carbamate

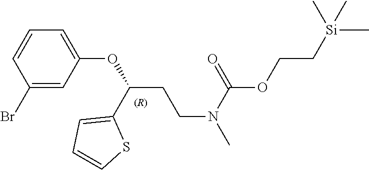

### Step 1: (R)-3-Chloro-1-(thiophen-2-yl)propan-1-ol

To a solution of (S)-Me-CBS oxazaborolidine (1 M (916 μl, 0.916 mmol) in dry toluene (50 mL), BH₃-DMS (2 M in toluene, 10.3 mL, 20.6 mmol) was added dropwise. After 10 min stirring the mixture was cooled to 0° C. and a solution of 3-chloro-1-(thiophen-2-yl)propan-1-one (prepared as described in intermediate 8, 2 g, 11.5 mmol) in dry toluene (60 mL) was added dropwise by maintaining the temperature below 0° C. The reaction mixture was allowed to stir at 0° C. for 1 h and then quenched by the addition of MeOH (10 mL) followed by 1 M HCl (10 mL). Water was added and the aqueous phase was extracted with DCM. The combined organic fractions were dried over Na₂SO₄, filtered and concentrated under reduced pressure. The residue was purified by combiflash chromatography (gold-column, CH/EtOAc up to 100%) giving the title compound (1.40 g, 69% yield).

### Step 2: (R)-3-(Methylamino)-1-(thiophen-2-yl)propan-1-ol

To a solution of the compound obtained in step 1 (1.61 g, 9.1 mmol) in abs. EtOH (12 mL) methylamine (40% solution in H₂O, 15 mL, 173 mmol) was added and the reaction mixture heated in a sealed tube at 130° C. for 1 h. Water was added to the mixture and EtOH was removed under reduced pressure. The aqueous phase was extracted with DCM. The combined organic fractions were dried over MgSO₄, filtered and the solvent removed under reduced pressure to afford the crude product which was recrystallized from methylcyclohexane/toluene (3:1), to give the title compound (0.861 g, 55% yield) as a white solid.

### Step 3: (R)-3-(3-Bromophenoxy)-N-methyl-3-(thiophen-2-yl)propan-1-amine

To a suspension of NaH (60% suspension in mineral oil, 402 mg, 10.1 mmol) in dry DMA (15 mL), a solution of the compound obtained in step 2 (861 mg, 5.03 mmol) in DMA (10 mL) was added, and the reaction mixture stirred at r.t. for 30 min. Then, 1-bromo-3-fluoro benzene (1.12 mL, 10.1 mmol) was added and the mixture was heated at 90° C. for 3 h. Water was added and the product extracted with AcOEt. The combined organic fractions were dried over Na₂SO₄, filtered and the solvent was removed under reduced pressure. The residue was purified by combiflash chromatography (SiO₂, CH/EtOAc up to 100%) to give the title compound (1.36 g, 83% yield).

### Step 4: Title Compound

To a solution of the compound obtained in step 3 (1.36 g, 4.17 mmol) in DCM (15 mL), DIPEA (726 μL, 4.17 mmol) was added followed by the dropwise addition of 4-nitrophenyl (2-(trimethylsilyl)ethyl) carbonate (1.18 g, 4.17 mmol) in DCM (5 mL). The reaction mixture was stirred for 16 h. Sat. aqueous NaHCO₃ solution was added to the mixture and the product extracted with DCM. The combined organic fractions were washed with 10% NaOH, dried over MgSO₄ and filtered. The solvent was removed under reduced pressure to give the title compound (1.78 g, 91% yield).

HPLC ret time (method A): 2.96 min; ESI−MS: m/z 468.4 [M−H]⁻.

### Intermediate 12: (S)-tert-butyl (3-(3-(2-chloropyrimidin-4-yl)phenoxy)-3-phenylpropyl)(methyl)carbamate

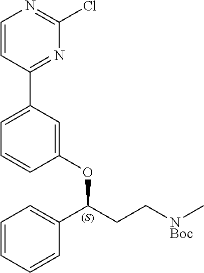

A Radley tube was charged with (S)-tert-butyl methyl(3-phenyl-3-(3-(4,4,5,5-tetramethyl-1,3,2-dioxaborolan-2-yl)phenoxy)propyl)carbamate (intermediate 4, 600 mg, 1.28 mmol), 2,4-dichloropyrimidine (191 mg, 1.28 mmol), PdCl₂(dppf)-CH₂Cl₂ (105 mg, 0.128 mmol) and purged with argon. Degassed Na₂CO₃ solution (0.4 M, 3.21 mL, 1.28 mmol) was added followed by toluene/ethanol (9/1, 20 mL) and the reaction mixture heated at 50° C. for 4 h under argon atmosphere. The layers were separated and the organic phase was washed with water and concentrated to dryness under reduced pressure. The residue was purified by combiflash chromatography (SiO₂, CH/EtOAc) to give the title compound (325 mg, 56% yield).

HPLC ret time (method A): 2.94 min; ESI+MS: 454.2 [M+H]⁺.

This method was used for the preparation of Intermediates 13-16, using suitable starting materials and Intermediates.

| INT | Structure | Chemical name |
|---|---|---|
| 13 | 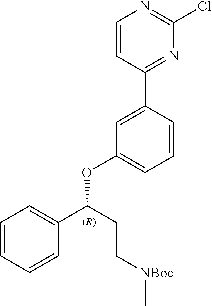 | (R)-tert-butyl (3-(3-(2- chloropyrimidin- 4-yl)phenoxy)-3- phenylpropyl) (methyl) carbamate |
| 14 | 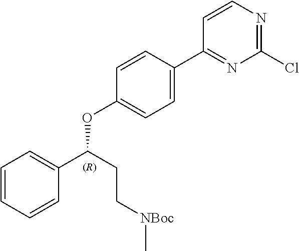 | (R)-tert-butyl (3-(4-(2- chloropyrimidin- 4-yl)phenoxy)-3- phenylpropyl) (methyl) carbamate |
| 15 | 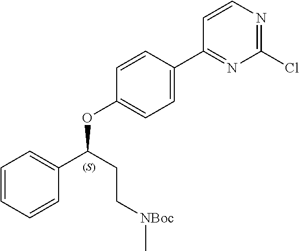 | (S)-tert-butyl (3-(4-(2- chloropyrimidin- 4-yl)phenoxy)-3- phenylpropyl) (methyl) carbamate |
| 16 | 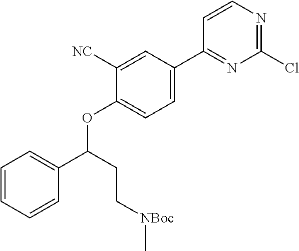 [SMILES: CN(CCC(Oc1ccc(-c2ccnc(Cl)n2)cc1C#N)c1ccccc1)C(=O)OC(C)(C)C] | tert-butyl (3-(4-(2- chloropyrimidin- 4-yl)-2- cyanophenoxy)- 3-phenylpropyl) (methyl) carbamate |

### SYNTHESIS OF EXAMPLES

### Examples 1: 3-(3-(2-Methoxypyrimidin-4-yl)phenoxy)-N-methyl-3-phenylpropan-1-amine

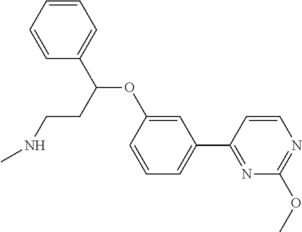 [SMILES: CNCCC(Oc1cccc(-c2ccnc(OC)n2)c1)c1ccccc1]

### Step 1: tert-Butyl (3-(3-(2-methoxypyrimidin-4-yl)phenoxy)-3-phenylpropyl)(methyl)carbamate

A Radley tube was charged with tert-butyl methyl(3-phenyl-3-(3-(4,4,5,5-tetramethyl-1,3,2-dioxaborolan-2-yl)phenoxy)propyl)carbamate (Intermediate 1, 200 mg, 0.163 mmol), 4-chloro-2-methoxypyrimidine (250 mg, 1.73 mmol), PdCl₂(dppf)-CH₂Cl₂ (13.3 mg, 0.016 mmol) and purged with argon. Degassed Na₂CO₃ solution (0.4 M, 0.41 mL, 0.163 mmol) was added followed by toluene/EtOH (9/1, 7 mL) and the reaction mixture heated at 90° C. for 16 h under argon atmosphere. The layers were separated and the organic phase was washed with water and concentrated to dryness under reduced pressure. The residue was purified by combiflash chromatography (neutral alumina, CH/EtOAc up to 100%) to give the title compound (28 mg, 38% yield).

### Step 2. Title Compound

To a solution of the compound obtained in step 1 (28 mg, 0.062 mmol) in DCM (5 mL) at r.t. zinc bromide (168 mg, 0.747 mmol) was added. The reaction mixture was stirred at r.t. for 2 days, after which it was quenched with few drops of water. The reaction mixture was adjusted to basic pH with aqueous NH₄OH solution, stirred for 15 min and extracted with DCM. The combined organic fractions were dried over MgSO₄, filtered and concentrated under reduced pressure to afford the title compound (20 mg, 92% yield).

HPLC ret time (method A): 1.52 min; ESI+MS: m/z 350.2 [M+H]⁺.

This method was used for the preparation of Examples 2-16, using suitable starting materials and intermediates

### Examples X

| | | | Ret | | |
|---|---|---|---|---|---|
| | | | time | MS | HPLC |
| EX | Structure | Chemical name | (min) | [M + H]⁺ | Method |
| 2 | 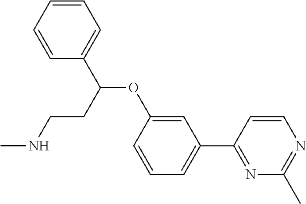 | N-methyl-3-(3-(2- methylpyrimidin-4- yl)phenoxy)-3- phenylpropan-1-amine | 1.43 | 334.2 | A |
| 3 | 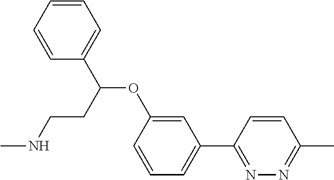 | N-methyl-3-(3-(6- methylpyridazin-3- yl)phenoxy)-3- phenylpropan-1-amine | 1.66 | 334.49 | C |
| 4 | 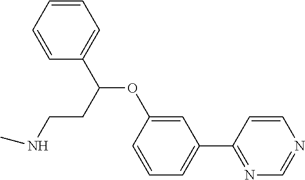 [SMILES: CNCCC(Oc1cccc(-c2ccncn2)c1)c1ccccc1] | N-methyl-3-phenyl-3- (3-(pyrimidin-4- yl)phenoxy)propan-1- amine | 1.37 | 320.2 | A |
| 5 | 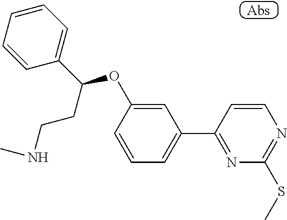 [SMILES: CNCC[C@H](Oc1cccc(-c2ccnc(SC)n2)c1)c1ccccc1] | (S)-N-methyl-3-(3-(2- (methylthio)pyrimidin-4- yl)phenoxy)-3- phenylpropan-1-amine | 2.06 | 366.13 | C |
| 6 | 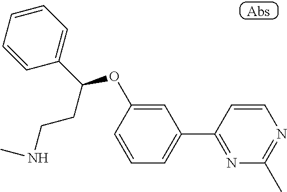 | (S)-N-methyl-3-(3-(2- methylpyrimidin-4- yl)phenoxy)-3- phenylpropan-1-amine | 1.45 | 334.2 | A |
| 7 | 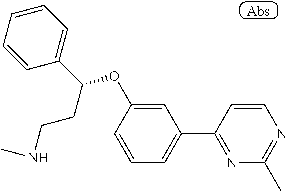 | (R)-N-methyl-3-(3-(2- methylpyrimidin-4- yl)phenoxy)-3- phenylpropan-1-amine | 1.45 | 334.2 | A |
| 8 | 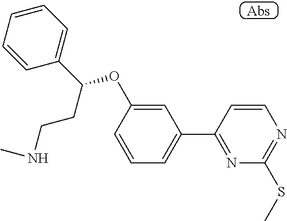 [SMILES: CNCC[C@@H](Oc1cccc(-c2ccnc(SC)n2)c1)c1ccccc1] | (R)-N-methyl-3-(3-(2- (methylthio)pyrimidin-4- yl)phenoxy)-3- phenylpropan-1-amine | 1.74 | 366.2 | A |
| 9 | 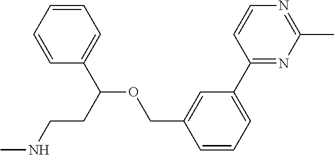 | N-methyl-3-((3-(2- methylpyrimidin-4- yl)benzyl)oxy)-3- phenylpropan-1-amine | 1.54 | 348.2 | A |
| 10 | 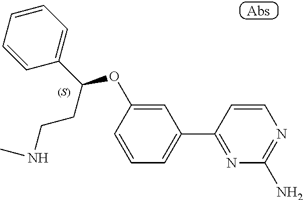 | (S)-4-(3-(3- (methylamino)-l- phenylpropoxy)phenyl) pyrimidin-2-amine | 1.34 | 335.2 | A |
| 11 | 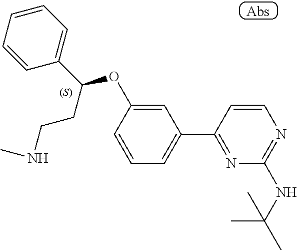 | (S)-N-(tert-butyl)-4-(3- (3-(methylamino)-1- phenylpropoxy)phenyl) pyrimidin-2-amine | 1.90 | 391.2 | A |
| 12 | 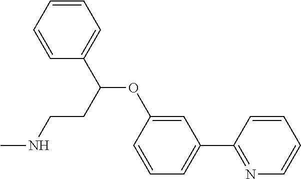 [SMILES: CNCCC(Oc1cccc(-c2ccccn2)c1)c1ccccc1] | N-methyl-3-phenyl-3- (3-(pyridin-2- yl)phenoxy)propan-1- amine | 1.62 | 319.1 | A |
| 13 | 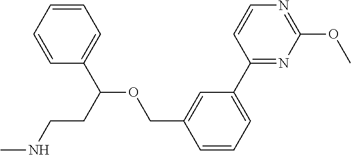 | 3-((3-(2- methoxypyrimidin-4- yl)benzyl)oxy)-N- methyl-3-phenylpropan- 1-amine | 1.67 | 364.2 | A |
| 14 | 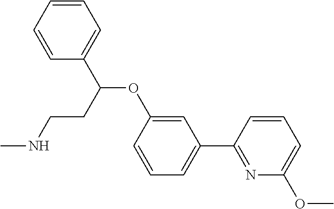 | 3-(3-(6-methoxypyridin- 2-yl)phenoxy)-N- methyl-3-phenylpropan- 1-amine | 1.97 | 349.2 | A |
| 15 | 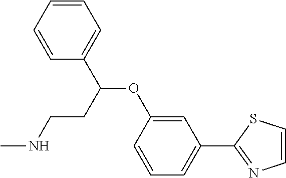 [SMILES: CNCCC(Oc1cccc(-c2nccs2)c1)c1ccccc1] | N-methyl-3-phenyl-3- (3-(thiazol-2- yl)phenoxy)propan-1- amine | 1.65 | 325.1 | A |
| 16 | 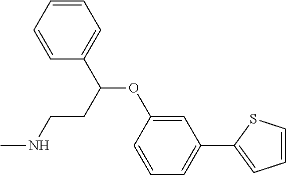 [SMILES: CNCCC(Oc1cccc(-c2cccs2)c1)c1ccccc1] | N-methyl-3-phenyl-3- (3-(thiophen-2- yl)phenoxy)propan-1- amine | 1.97 | 324.1 | A |

### Examples 17: N-Methyl-3-(3-(pyridin-3-yl)phenoxy)-3-(thiophen-2-ylpropan-1-amine

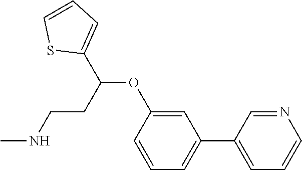 [SMILES: CNCCC(Oc1cccc(-c2cccnc2)c1)c1cccs1]

### Step 1: 2-(Trimethylsilyl)ethyl methyl(3-(3-(pyridin-3-yl)phenoxy)-3-(thiophen-2-yl)propyl)carbamate

A Radley tube was charged with 2-(trimethylsilyl)ethyl (3-(3-iodophenoxy)-3-(thiophen-2-yl)propyl)(methyl)carbamate (Intermediate 9, 116 mg, 0.224 mmol), pyridin-3-ylboronic acid (28 mg, 0.224 mmol), PdCl₂(dppf)-CH₂Cl₂ (18.3 mg, 0.022 mmol) and purged with argon. Aqueous Na₂CO₃ solution (0.4 M, 0.56 mL, 0.224 mmol) was added and the reaction mixture stirred at 90° C. overnight under argon atmosphere. The reaction mixture was concentrated and EtOAc and water were added to the mixture. The layers were separated and the organic phase was washed with water and concentrated to dryness. The residue was purified by combiflash chromatography (neutral alumina, CH/EtOAc up to 100%) to give the title compound (47 mg, 45 yield) as oil.

### Step 2: Title Compound

To a solution of the compound obtained in step 1 (47 mg, 0.10 mmol) in dry DMF (1 mL) cesium fluoride (76 mg, 0.501 mmol) was added. The reaction mixture was stirred at 60° C. for 6 h. The solvent was removed under reduced pressure and the residue was treated with aqueous NH₄OH and extracted with DCM. The combined organic fractions were dried MgSO₄, filtered and concentrated to afford the title compound (30 mg, 92% yield).

HPLC ret time (method A): 1.49 min; ESI+MS: m/z 325.1 [M+H]⁺.

This method was used for the preparation of Examples 18-27, using suitable starting materials:

### Examples X

| | | | Ret | | |
|---|---|---|---|---|---|
| | | | time | MS | HPLC |
| EX | Structure | Chemical name | (min) | [M + H]⁺ | Method |
| 18 | 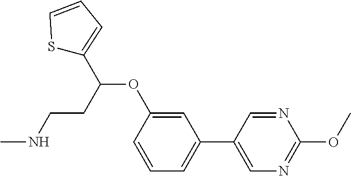 | 3-(3-(2- methoxypyrimidin-5- yl)phenoxy)-N-methyl- 3-(thiophen-2- yl)propan-1-amine | 1.49 | 325.1 | A |
| 19 | 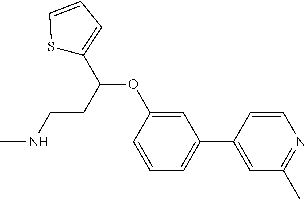 | N-methyl-3-(3-(2- methylpyridin-4- yl)phenoxy)-3- (thiophen-2-yl)propan- 1-amine | 1.47 | 339.1 | A |
| 20 | 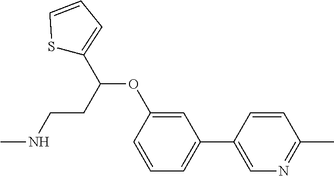 | N-methyl-3-(3-(6- methylpyridin-3- yl)phenoxy)-3- (thiophen-2-yl)propan- 1-amine | 1.51 | 339.1 | A |
| 21 | 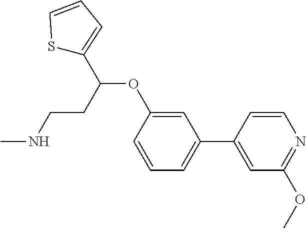 [SMILES: CNCCC(Oc1cccc(-c2ccnc(OC)c2)c1)c1cccs1] | 3-(3-(2-methoxypyridin- 4-yl)phenoxy)-N- methyl-3-(thiophen-2- yl)propan-1-amine | 1.65 | 355.1 | A |
| 22 | 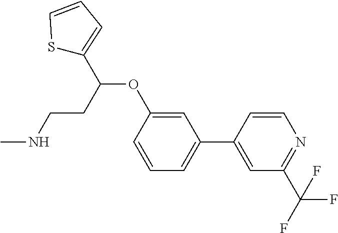 [SMILES: CNCCC(Oc1cccc(-c2ccnc(C(F)(F)F)c2)c1)c1cccs1] | N-methyl-3-(thiophen- 2-yl)-3-(3-(2- (trifluoromethyl)pyridin- 4-yl)phenoxy)propan-1 - amine | 1.79 | 393.1 | A |
| 23 | 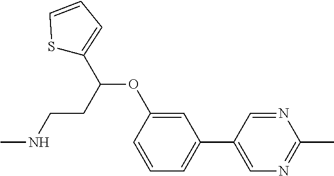 | N-methyl-3-(3-(2- methylpyrimidin-5- yl)phenoxy)-3- (thiophen-2-yl)propan- 1-amine | 1.37 | 340.0 | A |
| 24 | 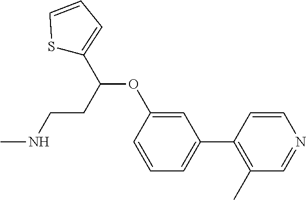 [SMILES: CNCCC(Oc1cccc(-c2ccncc2C)c1)c1cccs1] | N-methyl-3-(3-(3- methylpyridin-4- yl)phenoxy)-3- (thiophen-2-yl)propan- 1-amine | 1.54 | 339.0 | A |
| 25 | 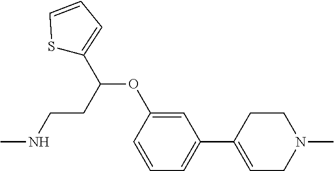 | N-methyl-3-(3-(1- methyl-1,2,3,6- tetrahydropyridin-4- yl)phenoxy)-3- (thiophen-2-yl)propan- 1-amine | 1.94 | 343.0 | A |
| 26 | 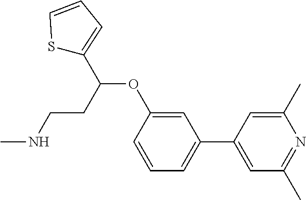 | 3-(3-(2,6- dimethylpyridin-4- yl)phenoxy)-N-methyl- 3-(thiophen-2- yl)propan-1-amine | 2.15 | 353.0 | A |
| 27 | 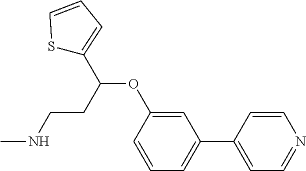 [SMILES: CNCCC(Oc1cccc(-c2ccncc2)c1)c1cccs1] | N-methyl-3-(3-(pyridin- 4-yl)phenoxy)-3- (thiophen-2-yl)propan- 1-amine | 1.49 | 325.1 | A |

### Example 28: (S)—N-Methyl-3-phenyl-3-(3-(2-(piperazin-1-yl)pyrimidin-4-yl)phenoxy)propan-1-amine

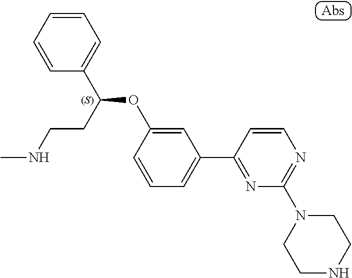

### Step 1: (S)-tert-Butyl 4-(4-(3-(3-((tert-butoxycarbonyl)(methyl)amino)-1-phenylpropoxy)phenyl)pyrimidin-2-yl)piperazine-1-carboxylate

A solution of (S)-tert-butyl (3-(3-(2-chloropyrimidin-4-yl)phenoxy)-3-phenylpropyl)(methyl)carbamate (Intermediate 12, 95 mg, 0.209 mmol) and tert-butyl piperazine-1-carboxylate (234 mg, 1.26 mmol) in NMP (2 mL) was microwave irradiated at 120° C. for 1 h. The reaction mixture was diluted with EtOAc and washed with water several times. The organic layer was dried MgSO₄, filtered and concentrated. The residue was purified by combiflash chromatography (neutral alumina, CH/EtOAc up to 100%) to give the title compound (103 mg, 82% yield).

### Step 2: Title Compound

To a solution of the compound obtained in step 1 (103 mg, 0.171 mmol) in DMF (5 mL) zinc bromide (461 mg, 2.05 mmol) was added and the reaction mixture was stirred at room temperature for 2 days. The solution was decanted and the insoluble part washed twice with DCM. The residue was then treated with aq. NH₄OH (until basic pH) for 10 min and extracted with DCM. The combined organic fractions were dried over MgSO₄, filtered and concentrated to afford the title compound (49 mg, 71% yield) as oil.

HPLC ret time (method C): 1.44 min; ESI+MS: m/z 404.4 [M+H]⁺.

This method was used for the preparation of Examples 29-48, using suitable starting materials.

| | | | Ret | | |
|---|---|---|---|---|---|
| | | | time | MS | HPLC |
| EX | Structure | Chemical name | (min) | (M + H) | Method |
| 29 | 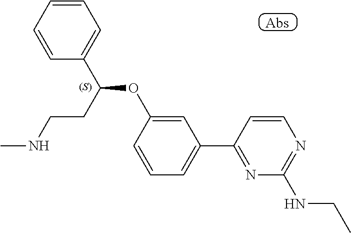 | (S)-N-ethyl-4-(3-(3- (methylamino)-1- phenylpropoxy)phenyl) pyrimidin-2-amine | 1.60 | 363.2 | A |
| 30 | 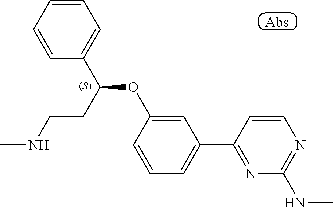 | (S)-N-methyl-4-(3-(3- (methylamino)-1- phenylpropoxy)phenyl) pyrimidin-2-amine | 1.48 | 349.2 | A |
| 31 | 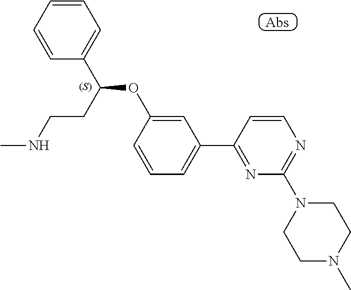 | (S)-N-methyl-3-(3-(2-(4- methylpiperazin-1- yl)pyrimidin-4- yl)phenoxy)-3- phenylpropan-1-amine | 1.63 | 418.3 | A |
| 32 | 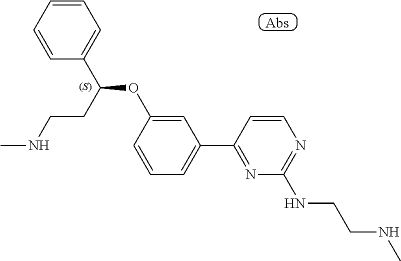 | (S)-N1 -methyl-N2-(4- (3-(3-(methylamino)-1- phenylpropoxy)phenyl) pyrimidin-2-yl)ethane- 1,2-diamine | 1.38 | 392.41 | C |
| 33 | 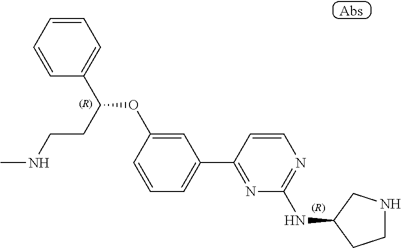 | 4-(3-((R)-3- (methylamino)-1- phenylpropoxy)phenyl)- N-((R)-pyrrolidin-3- yl)pyrimidin-2-amine | 1.4 | 404.52 | C |
| 34 | 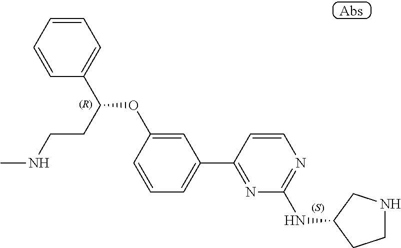 | 4-(3-((R)-3- (methylamino)-1- phenylpropoxy)phenyl)- N-((S)-pyrrolidin-3- yl)pyrimidin-2-amine | 1.38 | 404.7 | C |
| 35 | 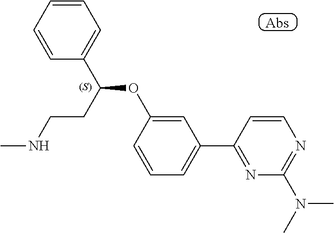 | (S)-N,N-dimethyl-4-(3- (3-(methylamino)-1- phenylpropoxy)phenyl) pyrimidin-2-amine | 1.74 | 363.3 | A |
| 36 | 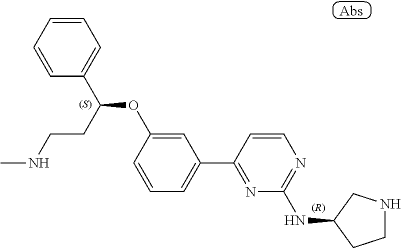 | 4-(3-{(S)-3- (methylamino)-1- phenylpropoxy)phenyl)- N-((R)-pyrrolidin-3- yl)pyrimidin-2-amine | 1.44 | 404.71 | C |
| 37 | 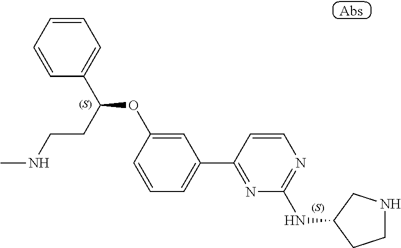 | 4-(3-((S)-3- (methylamino)-1- phenylpropoxy)phenyl)- N-((S)-pyrrolidin-3- yl)pyrimidin-2-amine | 1.47 | 404.43 | C |
| 38 | 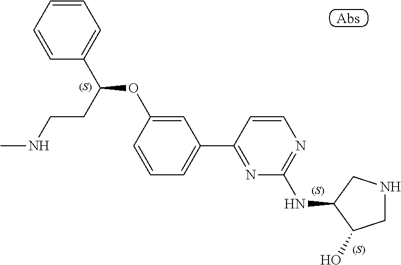 | (3S,4S)-4-((4-(3-((S)-3- (methylamino)-1- phenylpropoxy)phenyl) pyrimidin-2- yl)amino)pyrrolidin-3-ol | 1.18 | 420.3 | A |
| 39 | 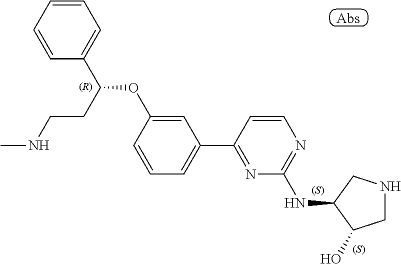 | (3S,4S)-4-((4-(3-((R)-3- (methylamino)-1- phenylpropoxy)phenyl) pyrimidin-2- yl)amino)pyrrolidin-3-ol | 1.6 | 420.57 | D |
| 40 | 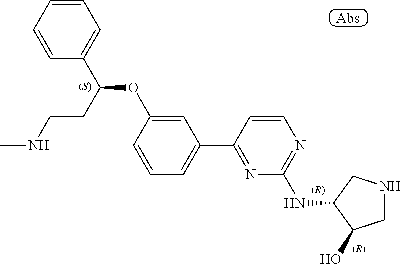 | (3R,4R)-4-((4-(3-((S)-3- (methylamino)-1- phenylpropoxy)phenyl) pyrimidin-2- yl)amino)pyrrolidin-3-ol | 1.33 | 420.32 | E |
| 41 | 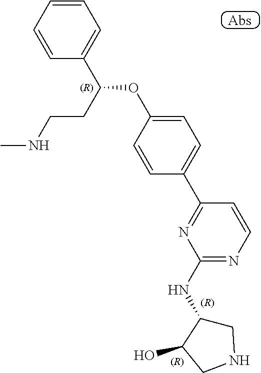 | (3R,4R)-4-((4-(4-({R)-3- (methylamino)-l- phenylpropoxy)phenyl) pyrimidin-2- yl)amino)pyrrolidin-3-ol | 1.31 | 420.34 | C |
| 42 | 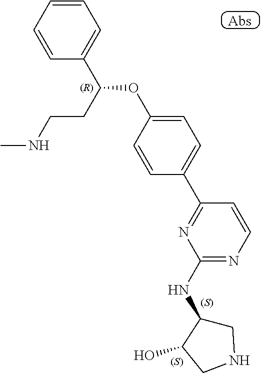 | (3S,4S)-4-((4-(4-((R)-3- (methylamino)-l- phenylpropoxy)phenyl) pyrimidin-2- yl)amino)pyrrolidin-3-ol | 1.43 | 420.4 | C |
| 43 | 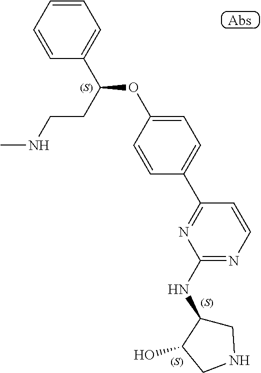 | (3S,4S)-4-((4-(4-((S)-3- (methylamino)-l- phenylpropoxy)phenyl) pyrimidin-2- yl)amino)pyrrolidin-3-ol | 1.28 | 420.34 | C |
| 44 | 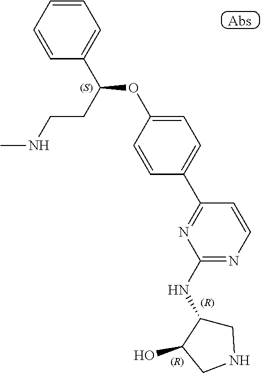 | (3R,4R)-4-((4-(4-((S)-3- (methylamino)-1- phenylpropoxy)phenyl) pyrimidin-2- yl)amino)pyrrolidin-3-ol | 1.28 | 420.32 | C |
| 45 | 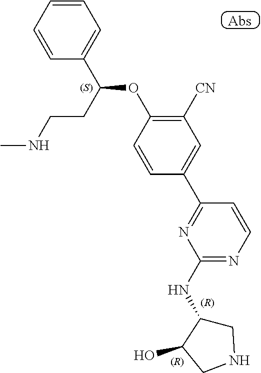 | 5-(2-(((3R,4R)-4- hydroxypyrrolidin-3- yl)amino)pyrimidin-4- yl)-2-((S)-3- (methylamino)-1- phenylpropoxy) benzonitrile | 1.87 | 445.19 | E |
| 46 | 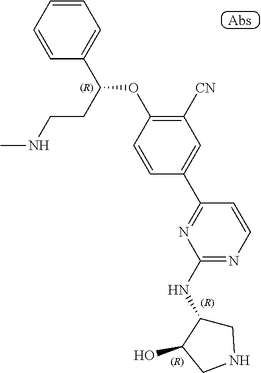 | 5-(2-(((3R,4R)-4- hydroxypyrrolidin-3- yl)amino)pyrimidin-4- yl)-2-((R)-3- (methylamino)-1- phenylpropoxy) benzonitrile | 1.71 | 445.2 | E |
| 47 | 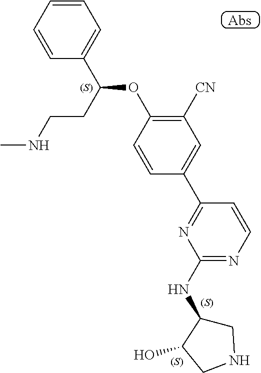 | 5-(2-(((3S,4S)-4- hydroxypyrrolidin-3- yl)amino)pyrimidin-4- yl)-2-((S)-3- (methylamino)-1- phenylpropoxy) benzonitrile | 1.91 | 445.24 | E |
| 48 | 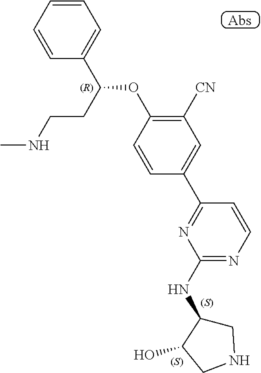 | 5-(2-(((3S,4S)-4- hydroxypyrrolidin-3- yl)amino)pyrimidin-4- yl)-2-((R)-3- l;methylamino)-1- phenylpropoxy) benzonitrile | 1.89 | 445.27 | E |

### Example 49: N-Methyl-3-(3-(2-methyl pyrimidin-4-yl)phenoxy)-3-(thiophen-2-yl)propan-1-amine

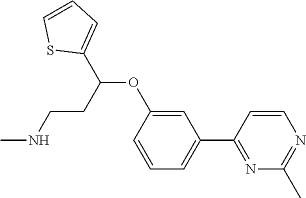

### Step 1: 2-(Trimethylsilyl)ethyl methyl(3-(3-(2-methylpyrimidin-4-yl)phenoxy)-3-(thiophen-2-yl)propyl)carbamate

A Radley tube was charged with 2-(trimethylsilyl)ethyl methyl(3-(3-(4,4,5,5-tetramethyl-1,3,2-dioxaborolan-2-yl)phenoxy)-3-(thiophen-2-yl)propyl)carbamate (Intermediate 8, 300 mg, 0.58 mmol), 4-chloro-2-methylpyrimidine (484 mg, 3.77 mmol), PdCl₂(dppf).CH₂Cl₂ (47.3 mg, 0.058 mmol) and purged with argon. Degassed aqueous Na₂CO₃ solution (0.4 M, 1.45 mL, 0.58 mmol) was added, followed by toluene/EtOH (9/1, 10 mL) and the reaction mixture stirred at 90° C. overnight under argon atmosphere. The reaction mixture was concentrated and EtOAc/water were added to the mixture. The layers were separated and the organic phase was washed with water and concentrated to dryness. The residue was purified by combiflash chromatography (neutral alumina, CH/EtOAc up to 100%) to give the title compound (43 mg, 15% yield) as oil.

### Step 2: Title Compound

To a solution of the compound obtained in step 1 (43 mg, 0.089 mmol) in dry DMF (3 mL) cesium fluoride (108 mg, 0.711 mmol) was added. The reaction mixture was stirred at 60° C. for 3 h. The solvent was removed under reduced pressure. The residue was treated with NH₄OH aq. and extracted with DCM. The combined organic fractions were dried MgSO₄, filtered and concentrated to afford the title compound (25 mg, 83% yield).

HPLC ret time (method A): 1.43 min; ESI+MS: m/z 340.1 [M+H]⁺.

This method was used for the preparation of Examples 50-52, using suitable starting materials.

| | | | Ret | | |
|---|---|---|---|---|---|
| | | | time | MS | HPLC |
| EX | Structure | Chemical name | (min) | [M + H]⁺ | Method |
| 50 | 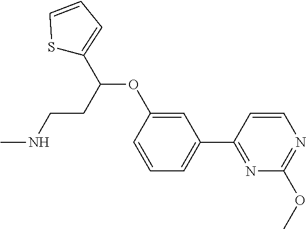 [SMILES: CNCCC(Oc1cccc(-c2ccnc(OC)n2)c1)c1cccs1] | 3-(3-(2- methoxypyrimidin-4- yl)phenoxy)-N-methyl- 3-(thiophen-2- yl)propan-1-amine | 1.56 | 356.1 | A |
| 51 | 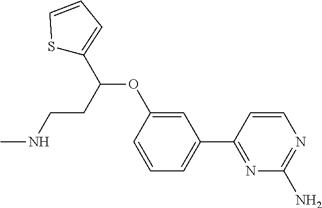 [SMILES: CNCCC(Oc1cccc(-c2ccnc(N)n2)c1)c1cccs1] | 4-{3-(3-{methylamino)- 1-(thiophen-2- yl)propoxy)phenyl)pyri midin-2-amine | 1.29 | 341.1 | A |
| 52 | 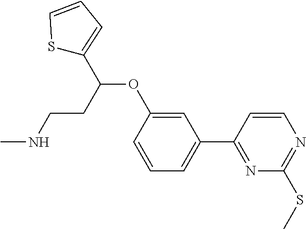 [SMILES: CNCCC(Oc1cccc(-c2ccnc(SC)n2)c1)c1cccs1] | N-methyl-3-(3-(2- (methylthio)pyrimidin-4- yl)phenoxy)-3- (thiophen-2-yl)propan- 1-amine | 1.69 | 372.1 | A |

### Example 53: (S)—N-Methyl-3-(3-(pyridin-4-yl)phenoxy)-3-(thiophen-2-yl)propan-1-amine

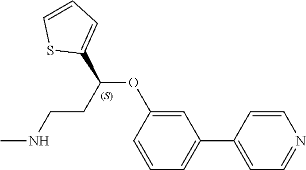

### Step 1: (S)-2-(Trimethylsilyl)ethyl methyl(3-(3-(pyridin-4-yl)phenoxy)-3-(thiophen-2-yl)propyl)carbamate

A Radley tube was charged with (S)-2-(trimethylsilyl)ethyl (3-(3-bromophenoxy)-3-(thiophen-2-yl)propyl)(methyl)carbamate (Intermediate 10, 60 mg, 0.128 mmol), 4-(4,4,5,5-tetramethyl-1,3,2-dioxaborolan-2-yl)pyridine (26 mg, 0.128 mmol), PdCl₂(dppf)-CH₂Cl₂ (10.4 mg, 0.013 mmol) and purged with argon. Degassed aqueous Na₂CO₃ solution (0.4 M, 319 μL, 0.128 mmol) was added, followed by toluene/EtOH (9/1, 6 mL) and the reaction mixture stirred at 90° C. for 16 h under argon atmosphere. The reaction mixture was concentrated and EtOAc and water were added to the mixture. The layers were separated and the organic phase was washed with water and concentrated to dryness. The residue was purified by combiflash chromatography (neutral alumina, CH/EtOAc up to 100%) to give the title compound (28 mg, 47% yield) as oil.

### Step 2: Title Compound

To a solution of the compound obtained in step 1 (28 mg, 0.06 mmol) in dry DMF (2 mL) cesium fluoride (91 mg, 0.6 mmol) was added. The reaction mixture was stirred at 60° C. for 2 h and the solvent was removed under reduced pressure. The residue was treated with NH₄OH aq. and extracted with DCM. The combined organic fractions were dried MgSO₄, filtered and concentrated to afford the title compound (19.4 mg, 99%% yield) as oil.

HPLC ret time (method A): 1.41 min; ESI+MS: m/z 325.1 [M+H]⁺.

This method was used for the preparation of Examples 54-57, using suitable starting materials.

| | | | Ret | | |
|---|---|---|---|---|---|
| | | | time | MS | HPLC |
| EX | Structure | Chemical name | (min) | [M + H]⁺ | Method |
| 54 | 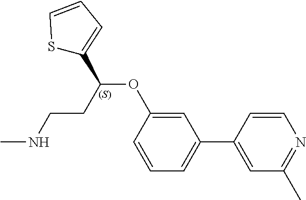 | (S)-N-methyl-3-(3-(2- methylpyridin-4- yl)phenoxy)-3- (thiophen-2-yl)propan- 1-amine | 2.11 | 339.0 | A |
| 55 | 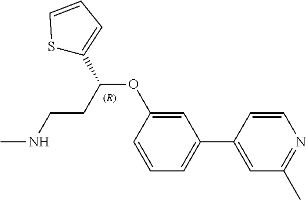 | (R)-N-methyl-3-(3-(2- methylpyridin-4- yl)phenoxy)-3- (thiophen-2-yl)propan- 1-amine | 1.56 | 339.0 | A |
| 56 | 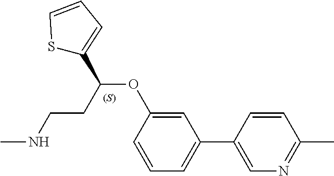 | (S)-N-methyl-3-(3-(6- methylpyridin-3- yl)phenoxy)-3- (thiophen-2-yl)propan- 1-amine | 1.92 | 339.0 | A |
| 57 | 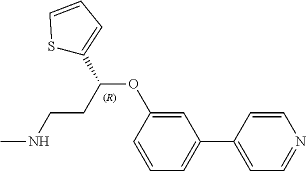 | (R)-N-methyl-3-(3- (pyridin-4-yl)phenoxy)- 3-(thiophen-2- yl)propan-1-amine | 1.50 | 325.0 | A |

### Example 58. (R)—N-Methyl-3-(3-(1-methylpiperidin-4-yl)phenoxy)-3-(thiophen-2-yl)propan-1-amine

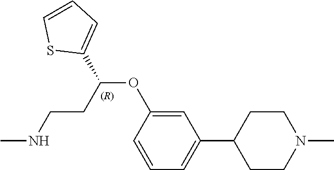

### Step 1: (R)-2-(Trimethylsilyl)ethyl (3-(3-(1-methyl-1,2,3,6-tetrahydropyridin-4-yl)phenoxy)-3-(thiophen-2-yl)propyl)carbamate

A Radley tube was charged with 2-(trimethylsilyl)ethyl (R)-2-(trimethylsilyl)ethyl (3-(3-bromophenoxy)-3-(thiophen-2-yl)propyl)carbamate (Intermediate 11, 300 mg, 0.638 mmol), 1-methyl-4-(4,4,5,5-tetramethyl-1,3,2-dioxaborolan-2-yl)-1,2,3,6-tetrahydropyridine (171 mg, 0.765 mmol), Pd(PPh₃)₄ (73.7 mg, 0.064 mmol), Na₂CO₃ (135 mg, 1.28 mmol) and purged with argon. A mixture of toluene/EtOH/water (6.5/1.5/1 mL) was added and the reaction mixture stirred at 80° C. for 16 h under argon atmosphere. The reaction mixture was evaporated to dryness and the residue purified by combiflash chromatography (gold column silica, CH/EtOAc up to 100% to give the title compound (184 m, 59% yield) as oil.

### Step 2: (R)-2-(Trimethylsilyl)ethyl methyl(3-(3-(1-methylpiperidin-4-yl)phenoxy)-3-(thiophen-2-yl)propyl)carbamate

The compound obtained in step 1 (130 mg, 0.267 mmol) in MeOH (15 mL) was hydrogenated (H-Cube, 70 mm cartridge, 10% Pd/C, 30 atm., 25° C., 1 mL/min) for 3 h. The solvent was removed under reduced pressure to give the title compound (75 mg, 57% yield).

### Step 3: Title Compound

To a solution of the compound obtained in step 2 (38 mg, 0.078 mmol) in dry DMF (1 mL) cesium fluoride (177 mg, 1.17 mmol) was added. The reaction mixture was stirred at 60° C. for 2 h. The solvent was removed under reduced pressure. The residue was treated with NH₄OH aq. and extracted with DCM. The combined organic fractions were dried MgSO₄, filtered and concentrated to afford the title compound (22 mg, 82% yield) as oil.

HPLC ret time (method A): 1.40 min; ESI+MS: m/z 345.1 [M+H]⁺.

### Example 59. N-Methyl-3-(3-(pyridin-2-yl)phenoxy)-3-(thiophen-2-yl)propan-1-amine

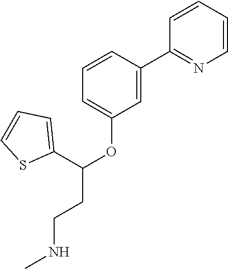 [SMILES: CNCCC(Oc1cccc(-c2ccccn2)c1)c1cccs1]

### Step 1: 2-(3-(3-Chloro-1-(thiophen-2-yl)propoxy)phenyl)pyridine

Following a similar method to that described in intermediate 8, step 3 and using 3-(pyridin-2-yl)phenol and 3-chloro-1-(thiophen-2-yl)propan-1-ol as starting materials, the title compound was obtained.

### Step 2: Title Compound

Following a similar method to that described in intermediate 8, step 4 and using the compound obtained in step 1 as starting material, the title compound was obtained.

HPLC ret time (method A): 1.61 min; ESI+MS: m/z 325.1 [M+H]⁺.

### Example 60: 4-(3-((R)-3-(Methylamino)-1-(thiophen-2-yl)propoxy)phenyl)-N—((S)-pyrrolidin-3-yl)pyrimidin-2-amine

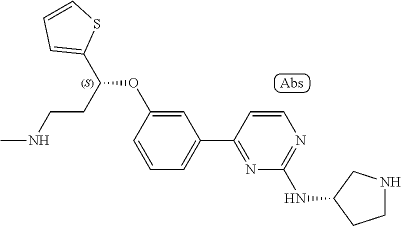

### Step 1: (R)-2-(Trimethylsilyl)ethyl (3-(3-(2-chloropyrimidin-4-yl)phenoxy)-3-(thiophen-2-yl)propyl)(methyl)carbamate

Following a similar method to that described in Example 49, step 1 and using (R)-2-(trimethylsilyl)ethyl methyl(3-(3-(4,4,5,5-tetramethyl-1,3,2-dioxaborolan-2-yl)phenoxy)-3-(thiophen-2-yl)propyl)carbamate and 2,4-dichloropyrimidine as starting materials, the title compound was obtained.

### Step 2: 2-(3-(3-Chloro-1-(thiophen-2-yl)propoxy)phenyl)pyridine

Following a similar method to that described in Example 28, step 1 and using the compound obtained in step 1 as starting material, the title compound was obtained.

### Step 3: Title Compound

Following a similar method to that described in Example 17, step 2 and using the compound obtained in step 2 as starting material, the title compound was obtained.

HPLC ret time (method F): 2.13 min; ESI+MS: m/z 410.17 [M+H]⁺.

Table of Examples with Binding to the Noradrenaline Transporter (NET) and the α₂δ-1 Subunit of the Voltage-Gated Calcium Channel:

### Pharmacological Study

Human α₂δ-1 Subunit of Caᵥ2.2 Calcium Channel Assay

Human α₂δ-1 enriched membranes (2.5 μg) were incubated with 15 nM of radiolabeled [3H]-Gabapentin in assay buffer containing Hepes-KOH 10 mM, pH 7.4. NSB (non specific binding) was measured by adding 10 μM pregabalin. After 60 min incubation at 27° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4. Filter plates were dried at 60° C. for 1 hour and 30 μL of scintillation cocktail were added to each well before radioactivity reading. Readings were performed in a Trilux 1450 Microbeta radioactive counter (Perkin Elmer).

### Binding Assay to Human Norepinephrine Transporter (NET).

Human norepinephrine transporter (NET) enriched membranes (5 μg) were incubated with 5 nM of radiolabeled [3H]-Nisoxetin in assay buffer containing 50 mM Tris-HCl, 120 mM NaCl, 5 mM KCl, pH 7.4.

NSB (non specific binding) was measured by adding 1 μM. After 60 min incubation at 4° C., binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, 0.9% NaCl, pH 7.4.

Filter plates were dried at 60° C. for 1 hour and 30 μL of scintillation cocktail were added to each well before radioactivity reading.

Readings were performed in a Trilux 1450 Microbeta radioactive counter (Perkin Elmer).

### Results:

This invention is aimed at providing a chemically related series of compounds which act as dual ligands of the α₂δ subunit of voltage-gated calcium channels and the NET receptor and especially compounds which have a binding expressed as Kᵢ responding to the following scales:

Kᵢ(NET) is preferably <1000 nM, more preferably <500 nM, even more preferably <100 nM.

Kᵢ(α₂δ-1) is preferably <10000 nM, more preferably <5000 nM, or even more preferably <500 nM.

The following scale has been adopted for representing the binding to the NET receptor expressed as Kᵢ:
+ Kᵢ-NET>=1000 nM
++ 500 nM<Ki-NET<1000 nM
+++ 100 nM<Ki-NET<500 nM
++++ Ki-NET<100 nM

The following scale has been adopted for representing the binding to the α₂δ-1 subunit of voltage-gated calcium channels expressed as Kᵢ:
+ Kᵢ(α₂δ-1)>=5000 nM
++ 500 nM<=Kᵢ(α₂δ-1)<5000 nM
+++ Kᵢ(α₂δ-1)<500 nM

All compounds prepared in the present application exhibit binding to the α₂δ-1 subunit of voltage-gated calcium channels and to the NET receptor, in particular the following binding results are shown:

| Example | Binding NET | Binding α2δ-1 |
|---|---|---|
| 1 | ++++ | ++ |
| 2 | +++ | ++ |
| 3 | ++ | + |
| 4 | ++ | ++ |
| 5 | ++++ | ++ |
| 6 | +++ | ++ |
| 7 | ++ | ++ |
| 8 | ++++ | ++ |
| 10 | +++ | ++ |
| 18 | + | ++ |
| 19 | +++ | +++ |
| 22 | +++ | ++ |
| 23 | + | ++ |
| 24 | ++ | ++ |
| 26 | +++ | ++ |
| 27 | +++ | ++ |
| 28 | + | ++ |
| 29 | +++ | ++ |
| 30 | ++++ | + |
| 31 | + | ++ |
| 32 | + | ++ |
| 33 | + | ++ |
| 34 | ++ | ++ |
| 35 | ++++ | + |
| 36 | ++ | ++ |

1-16. (canceled)

17. A compound of formula (I): 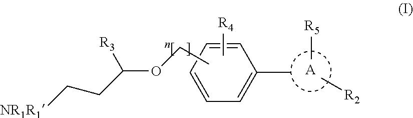
wherein
n is 0, 1, 2 or 3;
R₁ and R_{1′} are independently selected from the group consisting of hydrogen, halogen, substituted or unsubstituted C₁₋₆ alkyl, substituted or unsubstituted C₂₋₆ alkenyl, and substituted or unsubstituted C₂₋₆ alkynyl;
R₂ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl, substituted or unsubstituted heterocyclyl, substituted or unsubstituted alkylcycloalkyl, substituted or unsubstituted alkylaryl, substituted or unsubstituted alkylheterocyclyl, haloalkyl, —OR₆, —SR₆, and —NR₆R_{6′};
wherein R₆ and R_{6′} are independently selected from the group consisting of hydrogen, halogen, haloalkyl, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, —OR₈, and —NR₈R_{8′};
wherein R₈ and R_{8′} are independently selected from the group consisting of hydrogen, unsubstituted C₁₋₆ alkyl, unsubstituted C₂₋₆ alkenyl, and unsubstituted C₂₋₆ alkynyl;
R₃ is selected from the group consisting of substituted or unsubstituted aryl, substituted or unsubstituted alkylaryl, substituted or unsubstituted cycloalkyl, substituted or unsubstituted alkylcycloalkyl, substituted or unsubstituted heterocyclyl, and substituted or unsubstituted alkyheterocyclyl;
R₄ is selected from the group consisting of hydrogen, —CN, halogen, substituted or unsubstituted C₁₋₆ alkyl, substituted or unsubstituted C₂₋₆ alkenyl, substituted or unsubstituted C₂₋₆ alkynyl, OR₇, —NO₂, —NR₇R_{7′}, NR₇C(O)R_{7′}, —NR₇S(O)₂R_{7′}, —S(O)₂NR₇R_{7′}, —NR₇C(O)NR_{7′}R_{7″}, —SR₇, —S(O)R₇, S(O)₂R₇, —CN, haloalkyl, haloalkoxy, —C(O)OR₇, —C(O)NR₇R_{7′}, —OCH₂CH₂OH, —NR₇S(O)₂NR_{7′}R_{7″}, and C(CH₃)₂OR₇,
wherein R₇, R_{7′} and R_{7″}, are independently selected from hydrogen, unsubstituted C₁₋₆ alkyl, unsubstituted C₂₋₆ alkenyl, and unsubstituted C₂₋₆ alkynyl;
R₅ is selected from the group consisting of hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted alkylaryl, substituted or unsubstituted cycloalkyl, substituted or unsubstituted alkylcycloalkyl, substituted or unsubstituted heterocyclyl, substituted or unsubstituted alkyheterocyclyl, and haloalkyl;
cycle A is a heterocyclyl,
wherein cycle A is linked to the phenyl moiety of the compound formula (I) through a carbon atom and
wherein cycle A and the group 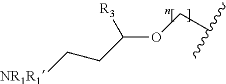
linked to the phenyl moiety of the compound of formula (I) stand in meta or para position to each other;
optionally as a stereoisomer, including enantiomers and diastereomers, a racemate or as a mixture of at least two of stereoisomers, including enantiomers and/or diastereomers, in any mixing ratio, or a corresponding salt thereof, or a corresponding solvate thereof.

18. The compound according to claim 17, wherein:
R₁ and R_{1′} are independently selected from the group consisting of hydrogen and substituted or unsubstituted C₁₋₆ alkyl.

19. The compound according to claim 17, wherein:
R₂ is selected from the group consisting of hydrogen, halogen, substituted or unsubstituted C₁₋₆ alkyl, substituted or unsubstituted heterocyclyl, haloalkyl, —OR₆, —SR₆, and —NR₆R_{6′};
wherein R₆ and R_{6′} are independently selected from the group consisting of hydrogen, halogen, substituted or unsubstituted C₁₋₆ alkyl, substituted or unsubstituted heterocyclyl, substituted or unsubstituted alkyheterocyclyl, OR₈, and —NR₈R_{8′},
wherein R₈ and R_{8′} are independently selected from the group consisting of hydrogen and unsubstituted C₁₋₆ alkyl.

20. The compound according to claim 19, wherein R₂ is NR₆R_{6′}, wherein R₆ and R_{6′} are independently selected from the group consisting of hydrogen, substituted or unsubstituted C₁₋₆ alkyl, substituted or unsubstituted heterocyclyl, and —NR₈R_{8′},
wherein R₈ and R_{8′} are independently selected from the group consisting of hydrogen and unsubstituted C₁₋₆ alkyl.

21. The compound according to claim 20, wherein R₆ is selected from the group consisting of hydrogen and substituted or unsubstituted C₁₋₆ alkyl.

22. The compound according to claim 20, wherein R_{6′} is selected from the group consisting of hydrogen, substituted or unsubstituted C₁₋₆ alkyl, and a substituted or unsubstituted N-containing heterocyclyl.

23. The compound according to claim 21, wherein R_{6′} is selected from the group consisting of hydrogen, substituted or unsubstituted C₁₋₆ alkyl, and a substituted or unsubstituted N-containing heterocyclyl.

24. The compound according to claim 17, wherein R₃ is selected from the group consisting of substituted or unsubstituted aryl, substituted or unsubstituted alkylaryl, substituted or unsubstituted cycloalkyl, substituted or unsubstituted alkylcycloalkyl, substituted or unsubstituted heterocyclyl, and substituted or unsubstituted alkyheterocyclyl.

25. The compound according to claim 24, wherein R₃ is selected from the group consisting of substituted or unsubstituted aryl and substituted or unsubstituted heterocyclyl.

26. The compound according to claim 25, wherein R₃ is selected from the group consisting of substituted or unsubstituted aryl and substituted or unsubstituted heteroaryl.

27. The compound according to claim 26, wherein R₃ is selected from the group consisting of substituted or unsubstituted phenyl and substituted or unsubstituted thiophene.

28. The compound according to claim 17, wherein Cycle A is a heteroaryl.

29. The compound according to claim 28, wherein Cycle A is an N-containing heteroaryl.

30. The compound according to claim 17, having the formula (Ia) or (Ib): 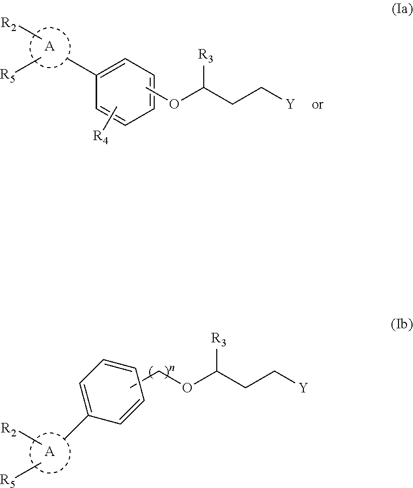
wherein Y represents NR₁R_{1′}, and R₁, R_{1′}, R₂, R₃, R₄, R₅, A and n are as defined in claim 17 for the compound of formula (I).

31. The compound according to claim 17, which is selected from the group consisting of:
3-(3-(2-Methoxypyrimidin-4-yl)phenoxy)-N-methyl-3-phenylpropan-1-amine,
N-methyl-3-(3-(2-methylpyrimidin-4-yl)phenoxy)-3-phenylpropan-1-amine,
N-methyl-3-(3-(6-methylpyridazin-3-yl)phenoxy)-3-phenylpropan-1-amine,
N-methyl-3-phenyl-3-(3-(pyrimidin-4-yl)phenoxy)propan-1-amine,
(S)—N-methyl-3-(3-(2-(methylthio)pyrimidin-4-yl)phenoxy)-3-phenylpropan-1-amine,
(S)—N-methyl-3-(3-(2-methylpyrimidin-4-yl)phenoxy)-3-phenylpropan-1-amine,
(R)—N-methyl-3-(3-(2-methylpyrimidin-4-yl)phenoxy)-3-phenylpropan-1-amine,
(R)—N-methyl-3-(3-(2-(methylthio)pyrimidin-4-yl)phenoxy)-3-phenylpropan-1-amine,
N-methyl-3-((3-(2-methylpyrimidin-4-yl)benzyl)oxy)-3-phenylpropan-1-amine,
(S)-4-(3-(3-(methylamino)-1-phenylpropoxy)phenyl)pyrimidin-2-amine,
(S)—N-(tert-butyl)-4-(3-(3-(methylamino)-1-phenylpropoxy)phenyl)pyrimidin-2-amine,
N-methyl-3-phenyl-3-(3-(pyridin-2-yl)phenoxy)propan-1-amine,
3-((3-(2-methoxypyrimidin-4-yl)benzyl)oxy)-N-methyl-3-phenylpropan-1-amine,
3-(3-(6-methoxypyridin-2-yl)phenoxy)-N-methyl-3-phenylpropan-1-amine,
N-methyl-3-phenyl-3-(3-(thiazol-2-yl)phenoxy)propan-1-amine,
N-methyl-3-phenyl-3-(3-(thiophen-2-yl)phenoxy)propan-1-amine,
N-Methyl-3-(3-(pyridin-3-yl)phenoxy)-3-(thiophen-2-yl)propan-1-amine,
3-(3-(2-methoxypyrimidin-5-yl)phenoxy)-N-methyl-3-(thiophen-2-yl)propan-1-amine,
N-methyl-3-(3-(2-methylpyridin-4-yl)phenoxy)-3-(thiophen-2-yl)propan-1-amine,
N-methyl-3-(3-(6-methylpyridin-3-yl)phenoxy)-3-(thiophen-2-yl)propan-1-amine,
3-(3-(2-methoxypyridin-4-yl)phenoxy)-N-methyl-3-(thiophen-2-yl)propan-1-amine,
N-methyl-3-(thiophen-2-yl)-3-(3-(2-(trifluoromethyl)pyridin-4-yl)phenoxy)propan-1-amine,
N-methyl-3-(3-(2-methylpyrimidin-5-yl)phenoxy)-3-(thiophen-2-yl)propan-1-amine,
N-methyl-3-(3-(3-methylpyridin-4-yl)phenoxy)-3-(thiophen-2-yl)propan-1-amine,
N-methyl-3-(3-(1-methyl-1,2,3,6-tetrahydropyridin-4-yl)phenoxy)-3-(thiophen-2-yl)propan-1-amine,
3-(3-(2,6-dimethylpyridin-4-yl)phenoxy)-N-methyl-3-(thiophen-2-yl)propan-1-amine,
N-methyl-3-(3-(pyridin-4-yl)phenoxy)-3-(thiophen-2-yl)propan-1-amine,
(S)—N-Methyl-3-phenyl-3-(3-(2-(piperazin-1-yl)pyrimidin-4-yl)phenoxy)propan-1-amine,
(S)—N-ethyl-4-(3-(3-(methylamino)-1-phenylpropoxy)phenyl)pyrimidin-2-amine,
(S)—N-methyl-4-(3-(3-(methylamino)-1-phenylpropoxy)phenyl)pyrimidin-2-amine,
(S)—N-methyl-3-(3-(2-(4-methylpiperazin-1-yl)pyrimidin-4-yl)phenoxy)-3-phenylpropan-1-amine,
(S)—N1-methyl-N2-(4-(3-(3-(methylamino)-1-phenylpropoxy)phenyl)pyrimidin-2-yl)ethane-1,2-diamine,
4-(3-((R)-3-(methylamino)-1-phenylpropoxy)phenyl)-N—((R)-pyrrolidin-3-yl)pyrimidin-2-amine,
4-(3-((R)-3-(methylamino)-1-phenylpropoxy)phenyl)-N—((S)-pyrrolidin-3-yl)pyrimidin-2-amine,
(S)—N,N-dimethyl-4-(3-(3-(methylamino)-1-phenylpropoxy)phenyl)pyrimidin-2-amine,
4-(3-((S)-3-(methylamino)-1-phenylpropoxy)phenyl)-N—((R)-pyrrolidin-3-yl)pyrimidin-2-amine,
4-(3-((S)-3-(methylamino)-1-phenylpropoxy)phenyl)-N—((S)-pyrrolidin-3-yl)pyrimidin-2-amine,
(3S,4S)-4-((4-(3-((S)-3-(methylamino)-1-phenylpropoxy)phenyl)pyrimidin-2-yl)amino)pyrrolidin-3-ol,
(3S,4S)-4-((4-(3-((R)-3-(methylamino)-1-phenylpropoxy)phenyl)pyrimidin-2-yl)amino)pyrrolidin-3-ol,
(3R,4R)-4-((4-(3-((S)-3-(methylamino)-1-phenylpropoxy)phenyl)pyrimidin-2-yl)amino)pyrrolidin-3-ol,
(3R,4R)-4-((4-(4-((R)-3-(methylamino)-1-phenylpropoxy)phenyl)pyrimidin-2-yl)amino)pyrrolidin-3-ol,
(3S,4S)-4-((4-(4-((R)-3-(methylamino)-1-phenylpropoxy)phenyl)pyrimidin-2-yl)amino)pyrrolidin-3-ol,
(3S,4S)-4-((4-(4-((S)-3-(methylamino)-1-phenylpropoxy)phenyl)pyrimidin-2-yl)amino)pyrrolidin-3-ol,
(3R,4R)-4-((4-(4-((S)-3-(methylamino)-1-phenylpropoxy)phenyl)pyrimidin-2-yl)amino)pyrrolidin-3-ol,
5-(2-(((3R,4R)-4-hydroxypyrrolidin-3-yl)amino)pyrimidin-4-yl)-2-((S)-3-(methylamino)-1-phenylpropoxy)benzonitrile,
5-(2-(((3R,4R)-4-hydroxypyrrolidin-3-yl)amino)pyrimidin-4-yl)-2-((R)-3-(methylamino)-1-phenylpropoxy)benzonitrile,
5-(2-(((3S,4S)-4-hydroxypyrrolidin-3-yl)amino)pyrimidin-4-yl)-2-((S)-3-(methylamino)-1-phenylpropoxy)benzonitrile,
5-(2-(((3S,4S)-4-hydroxypyrrolidin-3-yl)amino)pyrimidin-4-yl)-2-((R)-3-(methylamino)-1-phenylpropoxy)benzonitrile,
N-Methyl-3-(3-(2-methylpyrimidin-4-yl)phenoxy)-3-(thiophen-2-yl)propan-1-amine,
3-(3-(2-methoxypyrimidin-4-yl)phenoxy)-N-methyl-3-(thiophen-2-yl)propan-1-amine,
4-(3-(3-(methylamino)-1-(thiophen-2-yl)propoxy)phenyl)pyrimidin-2-amine,
N-methyl-3-(3-(2-(methylthio)pyrimidin-4-yl)phenoxy)-3-(thiophen-2-yl)propan-1-amine,
(S)—N-Methyl-3-(3-(pyridin-4-yl)phenoxy)-3-(thiophen-2-yl)propan-1-amine,
(S)—N-methyl-3-(3-(2-methylpyridin-4-yl)phenoxy)-3-(thiophen-2-yl)propan-1-amine,
(R)—N-methyl-3-(3-(2-methylpyridin-4-yl)phenoxy)-3-(thiophen-2-yl)propan-1-amine,
(S)—N-methyl-3-(3-(6-methylpyridin-3-yl)phenoxy)-3-(thiophen-2-yl)propan-1-amine,
(R)—N-methyl-3-(3-(pyridin-4-yl)phenoxy)-3-(thiophen-2-yl)propan-1-amine,
(R)—N-Methyl-3-(3-(1-methylpiperidin-4-yl)phenoxy)-3-(thiophen-2-yl)propan-1-amine,
N-Methyl-3-(3-(pyridin-2-yl)phenoxy)-3-(thiophen-2-yl)propan-1-amine, and
4-(3-((R)-3-(Methylamino)-1-(thiophen-2-yl)propoxy)phenyl)-N—((S)-pyrrolidin-3-yl)pyrimidin-2-amine.

32. A process for the preparation of the compound of formula (I) according to claim 17 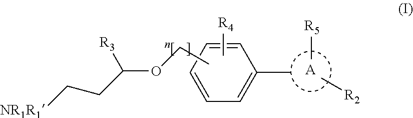
comprising:
treating a compound of formula (II) 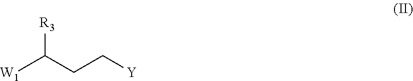
with a compound of formula (III): 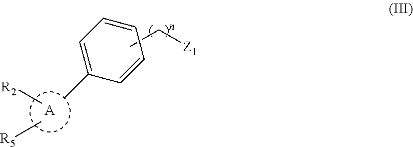
wherein
W₁ is OH or a leaving group, including halogen, mesylate, tosylate, nosylate, and triflate; Z₁ is OH or a leaving group, including halogen, mesylate, tosylate, nosylate, and triflate; Y represents NR₁R_{1′}, NHR₁P, or a leaving group, including halogen, mesylate, tosylate, nosylate, and triflate, P represents a protecting group, including tert-butoxycarbonyl, 2-(trimethylsilyl)ethoxycarbonyl and benzyl; R₁, R_{1′}, R₂, R₃, R₄, R₅, n, and A have the same meaning as defined in claim 17 for the compound of formula (I);
wherein
when W₁ is a leaving group, including halogen, mesylate, tosylate, nosylate and triflate, Z₁ is OH and n is 0.

33. A process for the preparation of a compound of formula (I) according to claim 17 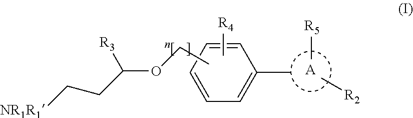
comprising:
treating a compound of formula (XI) 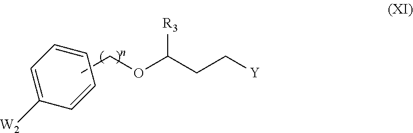
with a compound of formula (XIII) 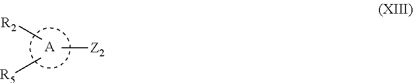
wherein
W₂ is BOM₂ or a leaving group, including halogen, mesylate, tosylate, nosylate, and triflate, wherein M represents hydrogen or alkyl or the two groups M, together with the boron atom form a cycle; Z₂ is OH or a leaving group, including halogen, mesylate, tosylate, nosylate, and triflate; Y represents NR₁R_{1′}, NHR₁P, or a leaving group, including halogen, mesylate, tosylate, nosylate, and triflate, P represents a protecting group, including tert-butoxycarbonyl, 2-(trimethylsilyl)ethoxycarbonyl, and benzyl; and R₁, R_{1′}, R₂, R₃, R₄, R₅, n and A have the same meaning as defined in claim 17 for the compound of formula (I).

34. A pharmaceutical composition which comprises the compound according to claim 17, or a pharmaceutically acceptable salt thereof, and a pharmaceutically acceptable carrier, adjuvant or vehicle.

35. A method of treating or preventing pain in a subject in need thereof, comprising administration of an effective amount of the compound according to claim 17.

36. The method according to claim 35, wherein the pain is selected from the group consisting of medium to severe pain, visceral pain, chronic pain, cancer pain, migraine, inflammatory pain, acute pain, neuropathic pain, allodynia, and hyperalgesia.
